(19) United States
(12) Patent Application Publication
(10) Pub. No.: US 2022/0145362 A1
(43) Pub. Date: May 12, 2022

(54) METHODS AND SYSTEMS FOR PROCESSING OR ANALYZING OLIGONUCLEOTIDE ENCODED MOLECULES

(71) Applicant: Haystack Sciences Corporation, South San Francisco, CA (US)

(72) Inventors: Richard Edward WATTS, Walnut Creek, CA (US); Divya KANICHAR, South San Francisco, CA (US); Patrick James MCENANEY, San Francisco, CA (US)

(73) Assignee: Haystack Sciences Corporation, South San Francisco, CA (US)

(21) Appl. No.: 17/438,900

(22) PCT Filed: Mar. 13, 2020
(86) PCT No.: PCT/US2020/022662

§ 371 Date: Sep. 13, 2021

Related U.S. Application Data
(60) Provisional application No. 62/818,645, filed on Mar. 14, 2019.

(51) Int. Cl. C12Q 1/6806; C12Q 1/686

(52) U.S. Cl. CPC C12Q 1/6806; C12Q 1/686

(57) ABSTRACT

The present disclosure provides methods and systems for determining a target-activity of at least one resolved oligonucleotide encoded molecule. In an embodiment, a method includes providing a separation medium, wherein the separation medium contains at least one target molecule; and various methods of separating a mixture of at least two oligonucleotide encoded molecules by electrophoresis based on different target-activities of the oligonucleotide encoded molecules for a target molecule. Benefits of the methods disclosed herein can include, without limitation, collecting and calculating qualitative and quantitative data for the target-activity of an encoded portion of the oligonucleotide encoded molecule for a target molecule.

### CROSS-REFERENCE TO RELATED APPLICATIONS

This application claims the benefit of U.S. Provisional Application No. 62/818,645, which was filed on Mar. 14, 2019, the entire contents of which is hereby incorporated by reference herein.

### BACKGROUND

Oligonucleotide encoded libraries can provide a useful method of directing the combinatorial synthesis of and identification of vast numbers of different molecules having different properties and reactivities. In general, an oligonucleotide encoded molecule can include an encoding portion, such as an oligonucleotide, tethered to an encoded portion. Typically, the encoding portion serves to either record or direct the combinatorial synthesis of the encoded portion and, after synthesis, serves to identify the structure of the encoded portion. By analogy, the encoding portion would be like a molecular barcode for a 3-D printer that tells the printer what to produce and then remains attached to identify the product after printing.

However, once a library of millions to trillions of different oligonucleotide encoded molecules has been synthesized, the challenge is to determine what useful properties those encoded portions might have. The synthesis of oligonucleotide encoded molecules and next-generation sequencing has been the subject of intense academic and industrial research, resulting in more and more efficient methods to synthesize oligonucleotide encoded molecules, to rapidly identify their encoded portions, and to determine their useful properties. Despite these advances, methods for separating, measuring, and/or determining the utility of the encoded portions of these molecules have failed to keep pace.

There remains a need to efficiently separate oligonucleotide encoded molecules based on their binding target-activity for target molecules. There remains a need to eliminate, or reduce, false positive and false negative results. There remains a need to provide qualitative and/or quantitative data regarding the target-activity of individual oligonucleotide encoded molecules for target molecules.

### SUMMARY

The present disclosure provides methods and systems for collecting target-activity data for at least one resolved oligonucleotide encoded molecule based at least in part on the differential target-activity of the oligonucleotide encoded molecule for a target molecule, as determined by electrophoresis and oligonucleotide sequencing. The present disclosure also provides methods and systems of separating a mixture of at least two oligonucleotide encoded molecules by electrophoresis based at least in part on different target-activities of the oligonucleotide encoded molecules for a target molecule. Benefits of the methods disclosed herein can include, for example, providing qualitative and quantitative data for the target-activity of an encoded portion of the oligonucleotide encoded molecule for a target molecule.

A method of determining a target-activity of at least one resolved oligonucleotide encoded molecule includes providing a separation medium, wherein the separation medium contains at least one target molecule; introducing a sample containing a mixture of at least two different oligonucleotide encoded molecules to the separation medium, wherein the at least two different oligonucleotide encoded molecules include an encoding portion operatively linked to at least one encoded portion; forming at least two different resolved oligonucleotide encoded molecules by separating the at least two different oligonucleotide encoded molecules into at least two separate locations in the separation medium; harvesting the at least one resolved oligonucleotide encoded molecule from the at least two different resolved oligonucleotide encoded molecules by segmenting at least one location of the at least two separate locations from the separation medium to form at least one resolved segment; processing the at least one resolved oligonucleotide encoded molecule to allow for performing polymerase chain reaction (PCR); amplifying the at least one encoded portion of the at least one resolved oligonucleotide encoded molecule by performing PCR on the encoding portion of the at least one resolved oligonucleotide encoded molecule; and determining a target-activity of the at least one resolved oligonucleotide encoded molecule by processing the at least one location and an identity of the at least one encoded portion of the at least one resolved oligonucleotide encoded molecule.

In an embodiment, the present method includes providing a separation medium, wherein the separation medium contains at least one target molecule; introducing a sample containing a mixture of at least two different oligonucleotide encoded molecules to the separation medium, wherein the at least two different oligonucleotide encoded molecules include an encoding portion operatively linked to at least one encoded portion; forming at least two different resolved oligonucleotide encoded molecules by separating the at least two different oligonucleotide encoded molecules into at least two separate locations in the separation medium; harvesting the at least one resolved oligonucleotide encoded molecule from the at least two different resolved oligonucleotide encoded molecules by segmenting at least one location of the at least two separate locations from the separation medium to form at least one resolved segment; processing the at least one resolved oligonucleotide encoded molecule to allow for PCR; amplifying the at least one encoded portion of the at least one resolved oligonucleotide encoded molecule by performing PCR on the encoding portion of the at least one resolved oligonucleotide encoded molecule; and collecting target-activity data for the at least one resolved oligonucleotide encoded molecule by correlating the at least one location with an identity of the at least one encoded portion of the at least one resolved oligonucleotide encoded molecule.

In an embodiment of the method, the at least one target molecule includes at least one of a cell, an oligonucleotide, a protein, an enzyme, a ribosome, and a nanodisc. In an embodiment of the method, the separation medium contains at least one of a particle, a polymer, and a separation surface, and the at least one target molecule is connected to at least one of the separation medium, the particle, the polymer, and the separation surface. In an embodiment of the method, the particle includes a polymer particle or a metal colloid. In an embodiment of the method, the polymer has a molecular weight of 10% or more of a lowest weight target molecule of the at least one target molecule. In an embodiment, the method includes separating the at least two different oligonucleotide encoded molecules based on at least one target-activity between the at least one target molecule and the encoded portion of the at least two different oligonucleotide encoded molecules. In an embodiment of the method, the at least one target-activity includes a chemical modification of the encoded portion of the at least one oligonucleotide encoded molecule by the at least one target molecule. In an embodiment of the method, the oligonucleotide contains at least two coding regions, the at least one encoded portion contains at least two positional building blocks, and each positional building block of the at least one encoded portion is identified by from 1 to 5 coding regions of the oligonucleotide. In an embodiment of the method, the separation medium contains a porous gel and a buffer system.

In an embodiment of the method, the at least two different oligonucleotide encoded molecules have a structure according to formula (I),

G-L-B (I)

wherein
G includes the oligonucleotide comprising at least two coding regions;
B is the encoded portion containing at least two positional building blocks;
L is a linker that operatively links G to B; and
wherein each positional building block in B is separately identified according to position by from 1 to 5 coding regions of G.

In an embodiment of the method, the at least two different oligonucleotide encoded molecules have a structure according to formula (II),

[(B₁)_{M}-L₁]_{O}-G-[(L₂-(B₂)_{K}]_{P} (II)

wherein
G includes the oligonucleotide comprising at least two coding regions;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions of G.

In an embodiment of the method, the at least two different oligonucleotide encoded molecules have a structure according to formula (III),

[(B₁)_{M}-L₁]_{O}-G′-[(L₂-(B₂)_{K}]_{P} (III)

wherein
G′ includes the oligonucleotide, G′ including comprising at least two coding regions and at least one hairpin;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G′;
L₂ is a linker that operatively links B₂ to G′;
O is an integer from zero to 5;
P is an integer from zero to 5;
provided that at least one of O and P is an integer from 1 to 5; and
wherein each positional building block B₁ at position M and/or B₂ at position K is identified by from 1 to 5 coding regions of G′.

In an embodiment, the method further includes separating the at least two different oligonucleotide encoded molecules into at least two separate locations in the separation medium by applying a first separation treatment across the separation medium in a first direction, wherein the first separation treatment includes a first voltage protocol and a first duration. In an embodiment, the method further includes harvesting the at least one resolved oligonucleotide encoded molecule by segmenting the at least one location from the separation medium in a first segmenting direction that is substantially perpendicular to the first direction to form the at least one resolved segment. In an embodiment, the method further includes separating the at least two different oligonucleotide encoded molecules into at least two separate locations of the separation medium by applying a second separation treatment across the separation medium in a second direction, wherein the second direction is substantially perpendicular to the first direction, wherein the second separation treatment includes a second voltage protocol and a second duration. In an embodiment, the method further includes harvesting the at least one resolved oligonucleotide encoded molecule by segmenting the at least one location from the separation medium in a second segmentation direction that is substantially perpendicular to the first segmentation direction to form the at least one resolved segment.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown.

FIG. 1 is a flow chart depicting an embodiment of the methods disclosed herein.

FIG. 2 is an illustration of an embodiment of methods disclosed herein.

FIG. 3 is an illustration of an embodiment of a method for molding electrophoretic channels for target-activity separations.

FIG. 4 is an illustration of an embodiment of a method of performing two-dimensional electrophoresis using two different separation mediums.

FIG. 5 is a chemical representation of a synthetic plan for fluorescently labeling oligonucleotide encoded molecules.

FIG. 6A shows chemical structures of a positive control compound.

FIG. 6B shows chemical structures of a positive control compound.

FIG. 6C shows chemical structures of a positive control compound.

FIG. 6D shows chemical structures of a positive control compound.

FIG. 6E shows chemical structures of a positive control compound.

FIG. 6F shows chemical structures of a positive control compound.

FIG. 6G shows chemical structures of a positive control compound.

FIG. 6H shows chemical structures of a positive control compound.

FIG. 6I shows chemical structures of a positive control compound.

FIG. 6J shows chemical structures of a positive control compound.

FIG. 6K shows chemical structures of a positive control compound.

FIG. 6L shows chemical structures of a positive control compound.

FIG. 6M shows chemical structures of a positive control compound.

FIG. 6N shows chemical structures of a positive control compound.

FIG. 6O shows chemical structures of a positive control compound.

FIG. 6P shows chemical structures of a positive control compound.

FIG. 7A contains graphs of polarized fluorescence of compounds based on concentration, wherein 702, 704, 706, 708, 710, 712, 714, 716, 718, 720, 722, 724, 726, 728, 730, 732, correspond to the positive control compounds of FIGS. 6A, 6B, 6C, 6D, 6E, 6F, 6G, 6H, 6I, 6J, 6K, 6L, 6M, 6N, 6O, 6P, respectively.

FIG. 7B contains graphs of polarized fluorescence of compounds based on concentration.

FIG. 8A is a chromatogram of a control compound as separated by the target activity separations protocol.

FIG. 8B is a chromatogram of a control compound as separated by the target activity separations protocol.

FIG. 8C is a chromatogram of a control compound as separated by the target activity separations protocol.

FIG. 8D is a chromatogram of a control compound as separated by the target activity separations protocol.

FIG. 9 is a chromatogram of a mixture of control compounds as separated by the target activity separations protocol.

FIG. 10 is a chromatogram of a mixture of control compounds as separated by the target activity separations protocol.

FIG. 11A is a chromatogram of a mixture of control compounds as separated by the target activity separations protocol.

FIG. 11B is a chromatogram of a mixture of control compounds as separated by the target activity separations protocol.

FIG. 12 is an illustration of computer system for implementing embodiments of the systems and methods disclosed herein.

### DETAILED DESCRIPTION

Unless otherwise noted, all measurements are in standard metric units.

Unless otherwise noted, the prefix “u” when used directly as a unit measurement means “micro” and is typically abbreviated “μ.” For example, “uL” stands for “microliter” or “4.”

Unless otherwise noted, all instances of the words “a,” “an,” or “the” can refer to one, or more than one, of the word that they modify.

Unless otherwise noted, the phrase “at least one of” means one or more than one or any combination of more than one of an object. For example, “at least one of H₁, H₂, and H₃” means H₁, H₂, or H₃, or any combination thereof.

Unless otherwise noted, the term “about” refers to ±10% of the non-percentage number that is described, rounded to the nearest whole integer. For example, about 100 mm, can include 90 to 110 mm. Unless otherwise noted, the term “about” refers to ±5% of a percentage number. For example, about 20% can include 15 to 25%. When the term “about” is discussed in terms of a range, then the term refers to the appropriate amount less than the lower limit and more than the upper limit. For example, from about 100 to about 200 mm can include from 90 to 220 mm.

Unless otherwise noted, the term “hybridize,” “hybridizing,” “hybridized,” and “hybridization” includes Watson-Crick base pairing, which includes guanine-cytosine and adenine-thymine (G-C and A-T) pairing for DNA and guanine-cytosine and adenine-uracil (G-C and A-U) pairing for RNA. Typically, these terms are used in the context of the selective recognition of a strand of nucleotides for a complementary strand of nucleotides, called an anti-codon or anti-coding region.

The phrases “selectively hybridizing,” “selective hybridization,” “selectively sorting,” and “selective recognition” refer to a selectivity of from 5:1 to 100:1 or more of a complementary oligonucleotide strand relative to a non-complementary oligonucleotide strand.

The term “oligonucleotide encoded molecule” refers to a molecule of the present disclosure that contains an oligonucleotide and at least one encoded portion.

The term “encoding portion” refers to a portion of an oligonucleotide encoded molecule that includes an oligonucleotide, wherein the oligonucleotide encodes and can identify the encoded portion of the oligonucleotide encoded molecule.

The term “encoded portion” refers to one or more parts of the oligonucleotide encoded molecule that contains a structure of building blocks, such as positional building blocks B₁ and B₂, which are encoded and can be identified by the encoding portion of the oligonucleotide encoded molecule. For example, the term “encoded portion” does not include, for example, a linker, even though these structures may be added as part of the process of synthesizing the encoded portion, because the linker was not encoded by the encoding portion of the oligonucleotide encoded molecule. As a second example, the terms “encoding portion” and “encoded portion” would not include molecular structures introduced after the encoding process, such as a fluorescent side chain.

The phrase “total number of positional building blocks” refers to an aggregate number of building blocks in an encoded portion. The meaning of the term “building block” can vary according to context. The term “building block” generally refers to a chemical change that is encoded in the encoding portion and which is made to an encoded portion. A first example of a building block is a chemical subunit which can be reacted with and bound to a linker or another building block to form part of an encoded portion. As a second example, a building block can be a chemical change that includes the removal of a chemical moiety. Specific examples of this include, but are not limited to, the hydrolysis of an ester, or the deprotection of an amine or aldehyde or alcohol. A third example includes building blocks representing chemical changes made to a linker or another building block that change the reactivity of the linker or the building block. Specific examples include but are not limited to the oxidation of an alcohol to an aldehyde or ketone, the reduction of an aldehyde or ketone to an alcohol, the reduction of a nitro group to an amine, the reduction of an azide to an amine, or the oxidation of an amine to a nitro group or an azide.

The terms “identified,” “identify,” and “identifies” refer to a correlation present between a coding region or a combination of coding regions of the encoding portion and the structure and/or sequence of building blocks of the encoded portion of the oligonucleotide encoded molecule. Generally, this correlation of sequence of a coding region can be combined with the knowledge of the synthetic steps used to construct the encoded portion to allow for the deduction or identification of the sequence, structure, and/or predicted structure of the encoded portion, even if and when the sequence is indirectly obtained from a PCR generated copy of the encoding portion of the oligonucleotide encoded molecule.

The terms “first,” “second,” etc. are understood to be terms that merely designate or distinguish which object is being referred to and are often based on a sequence of whichever one happens to be encountered first. For example, a “first” array is the array which happens to be used first and a first coding region is the first coding region that happens to be capable of being immobilized on the first array. Unless otherwise noted, the terms “first,” “second,” etc., do not refer to a position within the molecule. For example, it is understood that a first coding region and a second coding region may or may not be sequential and may or may not be close to one another within the encoding portion.

In the present disclosure, the hyphen or dashes in a molecular formula indicate that the parts of the formula are directly connected to each other through a covalent bond or hybridization.

Unless otherwise noted, all ranges of nucleotides, integer values, and percentages include all intermediate integer numbers as well as the endpoints. For example, the range of from 5 to 10 nucleotides would be understood to include 5, 6, 7, 8, 9, and 10 nucleotides.

In certain embodiments, the present disclosure relates to oligonucleotide encoded molecules (OEMs) that contain at least one oligonucleotide portion, as the encoding portion, and at least one encoded portion, wherein the oligonucleotide portion directed or encoded the synthesis of the at least one encoded portion using combinatorial chemistry. In certain embodiments, the oligonucleotide portion of the oligonucleotide encoded molecule can identify or facilitate the deduction of the at least one encoded portion of the oligonucleotide encoded molecule. In certain embodiments, an oligonucleotide encoded molecule of the present disclosure contains at least one oligonucleotide or oligonucleotide portion that contains at least two coding regions, wherein a combination of the at least two coding regions corresponds to and can be used to identify or deduce the sequence of building blocks in or structure of the encoded portion. In certain embodiments, the at least one oligonucleotide or oligonucleotide portion can be amplified by polymerase chain reaction (PCR) to produce copies of the at least one oligonucleotide or oligonucleotide portion. In an embodiment, the original oligonucleotide or oligonucleotide portion or copies thereof can be sequenced to determine the identity of a combination of at least two coding regions of the oligonucleotide encoded molecule. In certain embodiments, the identity of the combination of the at least two coding regions can be correlated to the series of combinatorial chemistry steps used to synthesize the encoded portion of the oligonucleotide encoded molecule. In certain embodiments, the series of combinatorial chemistry steps used to synthesize the encoded portion can identify or allow for the deduction of the encoded portion of the oligonucleotide encoded molecule.

Methods of synthesizing libraries of oligonucleotide encoded molecules have been, and continue to be, the focus of intense academic and industrial research due to their ability to systematically evaluate intermolecular properties, such as target-activity, of millions to billions of molecules for a target molecule. For example, if a target molecule is a key enzyme in a cancer pathway, then a researcher can determine if an encoded portion of one or more of millions to billions of oligonucleotide encoded molecules can bind to that enzyme in a way that increases, or decreases, its rate of catalyzing reactions. By analogy, this is like determining the combination to a digital lock by using a device that can electronically try millions of combinations in a short amount of time until the right combination is found. However, digital locks are designed to be unlocked by a correct sequence of digits, whereas a biological target may have no right combination of structures, but hopefully one or more molecules can be found that will work well enough to be commercialized as an effective therapy. To address this challenge, libraries of oligonucleotide encoded molecules can use a sort of guided evolution to get closer and closer to a molecule that may have desirable binding affinity. For example, if an oligonucleotide encoded molecule weakly reacts with a target molecule, then the next library of oligonucleotide encoded molecules can be synthesized to explore structural variations of the encoded portion of the most promising candidates, with the hope of finding an encoded portion with even better binding properties. This guided evolution and evaluation can be advanced until an effective solution is found, or a dead end is reached, such that the next molecule is selected as a starting point for guided evolution research.

However, despite the promise of this field of research for discovering molecules with useful properties, the vast majority of research has been on developing effective methods of synthesizing libraries of oligonucleotide encoded molecules. There has also been considerable development of next-gen methods of sequencing and identifying the encoded portion of an oligonucleotide encoded molecule from the encoding portion or a polymerase chain reaction (PCR) copy thereof. There has also been considerable effort to apply statistical techniques to wring greater dimensionality out of the sequencing data. There has been less progress on the problem of how the oligonucleotide encoded molecules are tested on target molecules for desirable properties.

One traditional method of testing if a library of oligonucleotide encoded molecules reacts with target molecules is the “mass exposure” method, which in certain cases is referred to as “panning”. The mass exposure method simply exposes a target molecule to a library of oligonucleotide encoded molecules, or a portion thereof, in a solvent or medium. Typically, the target molecule is immobilized, and the oligonucleotide encoded molecule binds the target molecule. After an exposure period, the solvent or medium is removed, leaving the strong binding oligonucleotide encoded molecules attached to, or associated with, the target molecule. These strong binding oligonucleotide encoded molecules can be identified by using PCR to make copies of the encoding portion followed by sequencing the copies, or originals, to decode and identify the structure of the strong binding encoded portions. This method can discover which members or particular oligonucleotide encoded molecules strongly bind to or associate with a target molecule. However, this mass exposure method is unreliable, and the suite of binders identified in replicate experiments can vary widely. Typically, only the strongest binders are reproducibly captured each time, even though there may be many molecules that are only moderate or weak binders but whose structures could provide valuable insight in correlating chemical structures to biological activities. Further, this method can provide false positives, where the member that remains immobilized in a testing chamber binds to some part of the testing environment, such as the chamber itself, or the tethering medium, or another oligonucleotide encoded molecule. The mass exposure method also provides false negatives, because molecules that weakly, moderately, or even strongly bind the target molecules can bind, unbind, and then be washed away when the solvent is removed. Molecules that bind, unbind and are washed away can never be recovered or identified in the sequencing data, and thus, this kind of false negative that is due to assay attrition renders real binders indistinguishable from non-binders. A second source of false negatives is the presence of many false positives. Often, false positives give signals in sequencing data that are stronger than real binders. In such a case, one cannot discern the real binder from the false positive, and the real binder is lost in the noise. In fact, this mass exposure method provides no data for measuring target affinity of an oligonucleotide encoded molecule for a target molecule. The data produced by this standard method is binary: present during PCR and sequencing means bound; and not present during PCR means not bound. The mass exposure method may be efficient from a processing point of view, but it is inefficient and limited from a data acquisition point of view. Because acquiring data is the primary goal of high-throughput screening of libraries of oligonucleotide encoded molecules, the mass exposure method has become a bottle neck in the drug discovery process.

Another traditional method of testing if a library of oligonucleotide encoded molecules binds a target molecule is the “mass exposure then electrophoresis” method, in some cases referred to as a “gel shift assay.” In this method, the mass exposure then electrophoresis method simply exposes a target molecule to a library of oligonucleotide encoded molecules in a solvent or medium in a manner similar to that of the “mass exposure” method previously discussed, except the target molecule is not bound. Instead, the mixture of a library of oligonucleotide encoded molecules bound to target molecules, unbound target molecules, and unbound oligonucleotide encoded molecules is purified by subjecting the mixture to traditional electrophoresis. The traditional electrophoresis separates the oligonucleotide encoded molecules bound to target molecules based on differences between the size and charge of the molecules, which may separate target molecules bound to oligonucleotide encoded molecules from unbound target molecules and unbound oligonucleotide encoded molecules. This method of mass exposure followed by electrophoresis may have the benefit of separating those oligonucleotide encoded molecules bound to a target molecule from those oligonucleotide encoded molecules and target molecules that remain unbound. However, this conventional technique sufferers the same false negatives, and provides the same binary data: bound or unbound.

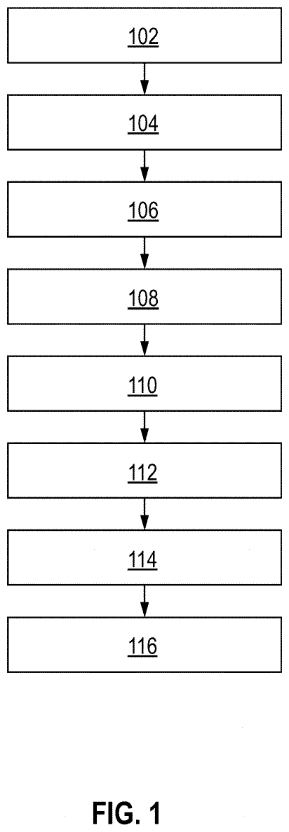

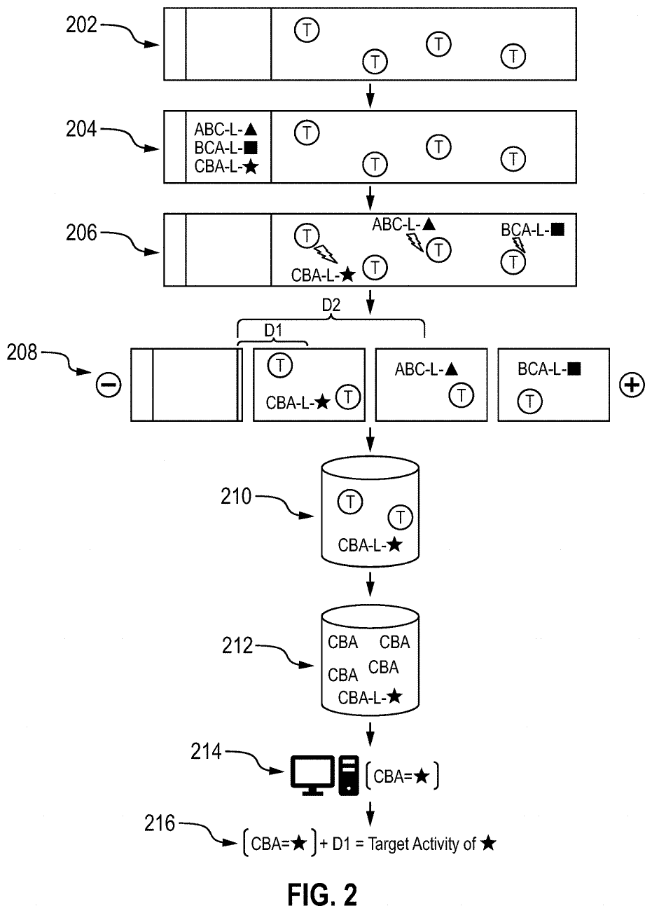

The present disclosure relates to a method of separating oligonucleotide encoded molecules by applying a type of “target-activity electrophoresis.” As a general overview of the method disclosed herein, referring to FIG. 1 and FIG. 2, the method includes providing a separation medium, wherein the separation medium contains at least one target molecule 102, 202; introducing a sample containing a mixture of at least two different oligonucleotide encoded molecules to the separation medium, wherein the at least two different oligonucleotide encoded molecules include an encoding portion (e.g., “CBA”) operatively linked (e.g. “L”) to at least one encoded portion (“star shape”) 104, 204; forming at least two different resolved oligonucleotide encoded molecules by separating the at least two different oligonucleotide encoded molecules into at least two separate locations in the separation medium 106, 206 (relative target activity depicted by size of lightning bolt symbol); harvesting the at least one resolved oligonucleotide encoded molecule from the at least two different resolved oligonucleotide encoded molecules by segmenting at least one location of the at least two separate locations from the separation medium to form at least one resolved segment and measuring the migration distance 108, 208 (depicted as D1 or D2); processing the at least one resolved oligonucleotide encoded molecule to allow for PCR 110, 210; amplifying the at least one encoded portion of the at least one resolved oligonucleotide encoded molecule by performing PCR on the encoding portion of the at least one resolved oligonucleotide encoded molecule 112, 212; sequencing the encoding portion of a resolved oligonucleotide encoded molecule, or a PCR copy thereof, and identifying the at least one encoded portion of the at least one resolved oligonucleotide encoded molecule 114, 214; collecting target-activity data for the at least one resolved oligonucleotide encoded molecule by correlating the at least one location with an identity of the at least one encoded portion of the at least one resolved oligonucleotide encoded molecule 116, 216.

Generally, the method includes introducing a mixture of oligonucleotide encoded molecules to a separation medium containing target molecules and using electrophoresis to migrate the oligonucleotide encoded molecules through the separation medium containing target molecules such that the oligonucleotide encoded molecules are separated, at least in part, on the basis of activity between the target molecule and the encoded portion of the oligonucleotide encoded molecules. Then, in an embodiment, the method can include segmenting the separation medium containing the separated or resolved oligonucleotide encoded molecules and measuring the migration distance of the different segments from the starting point or sample well. Then, in an embodiment, the method can include harvesting the oligonucleotide encoded molecules by processing the separation medium to allow for PCR amplification of the encoding portion of the oligonucleotide encoded molecules. In an embodiment, the method can include performing PCR amplification of the encoding portion of the oligonucleotide encoded molecules and sequencing, then correlating the sequence data to identify the encoded portion of oligonucleotide encoded molecules, sequencing the encoding portion, and identifying or deducting the structure of the encoded portion of the oligonucleotide encoded molecule. In an embodiment, the method can include collecting target-activity data by correlating identities of encoded portions to their migration distance or location in the separation medium.

In more detail, in an embodiment, the method can immobilize or reduce the mobility of a target molecule in a medium. In an embodiment, a library of oligonucleotide encoded molecules can be introduced to the separation medium at a sample well. Then the library of oligonucleotide encoded molecules can be subjected to electrophoresis, causing the library of oligonucleotide encoded molecules to migrate though the medium into contact with the immobilized target molecule. Further, in an embodiment, the library of oligonucleotide encoded molecules can be separated or resolved, in part, based on their activity with a target molecule. In an embodiment, oligonucleotide encoded molecules having a high activity with the target molecule will have their migration through the separation medium slowed, whereas oligonucleotide encoded molecules having a low activity with the target molecule will have their migration slowed less. In an embodiment, once sufficient separation has been achieved, portions of the sample mixture containing oligonucleotide encoded molecules can be recovered by segmenting the medium into portions. In an embodiment, those portions can be isolated by dissolving them in a solvent or soaking them in a solvent to allow the oligonucleotide encoded molecules in that portion to pass into the solvent. In an embodiment, once a portion of oligonucleotide encoded molecules has been recovered, the encoded portions of the oligonucleotide encoded molecule can be determined by performing PCR to form copies of the encoded portion of the oligonucleotide encoded molecules. In an embodiment, the copies of the encoded portion of the oligonucleotide encoded molecules can be sequenced, and the sequence data can be used to identify the oligonucleotide encoded molecule and measure the distance of migration of the oligonucleotide encoded molecule in the separation medium.

In an embodiment, one benefit of this method can be that false negatives are avoided or reduced, because even strong binding or reacting oligonucleotide encoded molecules can be recovered by applying stronger voltages so that deductions do not have to be made based the disappearance of molecules from detection. In an embodiment, a benefit of this method can be that measurements of migration distance for an oligonucleotide encoded molecule can provide qualitative and/or quantitative data for the affinity of an oligonucleotide encoded molecule for a target molecule. In contrast, conventional methods can only provide binary data for each molecule: binding or non-binding. In an embodiment, a benefit of the presently disclosed method can include measuring different types of interactions. For example, conventional methods can only measure probe-target affinity. In contrast, the methods disclosed herein can provide data regarding the chemical reactivity of an encoded portion of an oligonucleotide encoded molecule, because the chemical reactivity of a target molecule reacting with the encoded portion, e.g. catalyzing a reaction, tends to slow the rate of migration through the separation medium.

In an embodiment, the presently disclosed method can tremendously increase the number of real binders that are captured and identified. First, real binders are far less likely to be lost to assay attrition because there are no ‘washing’ steps. A washing step in the mass exposure/panning method uses the flow of liquid and is intended to move molecules that cannot bind the target away from the target. However, the real effect is to move molecules that are not bound—that is, washing will equally move both (a) molecules that cannot bind and (b) molecules that are only temporarily unbound. In contrast, the methods disclosed herein use electrophoresis to move molecules that are not bound to the target, but it moves them from one place where there is target to another place where there is target; this gives molecules that are only temporarily unbound greater opportunity to re-bind. Because this process is repeated many times along the path of migration, far fewer compounds are lost as false negatives to assay attrition. Second, because the voltage in the system places a very strong and continuous force on molecules that bind in transient, non-specific ways, these molecules are more thoroughly removed than they would be by washing processes. By virtue of being more completely removed, these compounds produce smaller signals in sequencing data and therefore are less likely to drown out the signal of real binders. The net effect is a very large increase in the signal to noise ratio, and a very large increase in the number of compounds that are identified as real binders.

In an embodiment, the method includes providing a separation medium, wherein the separation medium contains at least one target molecule. In an embodiment, the separation medium is not generally limited, so long as the medium allows for electrophoresis of oligonucleotide encoded molecules. Suitable separation mediums include a porous gel and a buffer system suitable for electrophoresis. Suitable porous gels can include an agarose, a polyacrylamide, various hydrogels, and starches. Suitable buffer systems can include Tris/Acetate/EDTA (TAE), Tris/Borate/EDTA (TBE), Tris/Borate (TB), and Lithium/Borate (LB), where EDTA stands for ethylenediaminetetraacetic acid and Tris stands for tris(hydroxymethyl)aminomethane. Porosity of the gel can be controlled utilizing various concentrations of the gelling material in the selected buffer system.

In an embodiment, the method includes providing a separation medium containing at least one target molecule. In an embodiment, a target molecule can be immobilized, or the mobility of a target molecule can be reduced by binding or tethering the target molecule to at least one of the separation medium, the particle, the polymer, and the separation surface. In an embodiment, the target molecule can be bound or tethered to at least one of the particle, the polymer, and the separation surface before, during, or after contacting the target with the separation medium. In an embodiment, a target molecule is bound or tethered to a particle before addition to a separation medium, such as an agarose gel. Generally, any suitable method of binding one molecule to a surface or other molecule can be used so long as the bond is stable during electrophoresis.

In an embodiment, the target molecule, such as a protein or protein complex, can be bound to an anchor including the separation medium, the particle, the polymer, and the separation surface using various binding methods known in the art. Suitable binding methods include amide bond cross-linking, sulfamide or sulfone formation, weakly reactive electrophilic interactions, polymerization reactions, disulfide formation, ester formation, click reactions (such as azide alkyne reactions with copper), Diels-Alder cycloadditions, and cross metathesis, calcium alginate immobilization through matrix trapping, and the like.

Immobilization of molecules, such as target molecules, onto solid surfaces is known to cause, or at least risk, deforming the molecule, which can hide the activity of the native molecule. Methods that avoid or reduce deformation of the target molecule can be advantageous, because they allow for the native activity of the target molecule to be measured. In an embodiment, the target molecule is attached, bound, strongly associated, or tethered to a polymer or an oligomer (other than the polymer and/or oligomer of the separation medium), wherein the polymer or oligomer has a molecular weight of 10% or more, including 20% to 5000%, of a lowest molecular weight of the target molecule. In an embodiment, a benefit of tethering the target molecule to a polymer or oligomer can be that the combination of oligonucleotide encoded molecule, target molecule, and polymer or oligomer can migrate at different rates through the separation medium, allowing for further separation from other molecules, while eliminating or reducing the risk of deforming the target molecule. In an embodiment, a benefit of tethering the target molecule to a polymer or oligomer can be that the oligonucleotide encoded molecule, target molecule, and polymer or oligomer can be removed from the separation medium into, for example, a solvent or buffer, prior to PCR and sequencing the encoding portion to identify the encoded portion.

In an embodiment, the method includes a separation medium, wherein the separation medium contains a target molecule bound or tethered to a particle. In an embodiment, the particle can be a composition labeled with an appropriate anti-target tag. In an embodiment, the particle can be a solid particle, an amorphous particle, a porous particle, a polymeric particle, a metal colloid, a mixture of materials, or a monomeric electrophoresis medium. Suitable particles can include an ion exchange resin, a silica particle, a polystyrene, an agarose bead, a biotin-labeled agarose, SEPHAROSE® beads, TENTAGEL® resin beads, dendrimeric polymers (polyethylene glycol, polystyrene, and the like), DYNABEADS® (magnetic particles); and calcium alginate immobilization through matrix trapping. In an embodiment, one benefit of binding or tethering a target molecule to a particle can be that the target molecule is immobilized or its migration during electrophoresis is slowed relative to the unbound target molecule.

In an embodiment, a benefit to attaching or tethering a target molecule to a particle can be that the particle is immobilized in the separation medium. In an embodiment, the target molecule is attached to or immobilized on a surface of a gel electrophoresis plate, where a gel electrophoresis plate is a surface on which the gel is formed. In an embodiment, a benefit to attaching or tethering a target molecule to a particle or surface can be that the migration of the target molecule is limited, or prevented, such that the migration rate of the target molecule is removed as a basis for separation.

Methods that can selectively bind the target molecule to a separation medium, the particle, the polymer, or the separation surface can be advantageous, because they allow for different binding mechanisms to be used, which can remove the choice of binding mechanism from consideration over multiple experiments. In an embodiment, a conjugate pair reaction binds a tagged target molecule selectively to the separation medium, the particle, the polymer, or the separation surface. Suitable conjugate pair reactions include a His tag, where His is histidine, in an integer between 6-10 to particles containing, or displaying on their surface a Nickel NTA, or Anti-His antibody; a biotin tag to particles containing a streptavidin, avidin or anti-biotin antibody; a streptavidin binding peptide to particles containing streptavidin or avidin; a halo-Tag to particles displaying the Halo-Tag protein; a FLAG tag to particles containing or displaying an anti-FLAG antibody; a calmodulin Binding protein to particles containing or displaying calmodulin; a glutathione S-Transferase to particles containing glutathione; a cellulose binding domain (CBP) to Cellulose particles or the separation medium; a native protein to particles containing or displaying an anti-protein antibody or covalently tethered to particles by surface lysine moieties reacted with carboxyl groups on the particle surface by common attachment chemistries (e.g., N-hydroxy succidimide or carbodiimide chemistry); a streptavidin protein to particles containing or displaying biotin or anti-streptavidin antibody; and an oligonucleotide labeled protein to particles containing or displaying the complimentary oligonucleotide.

In an embodiment, the method includes a target molecule in the separation medium. In an embodiment, the method can include adding or mixing a target molecule into the separation medium before a sample or mixture of oligonucleotide encoded molecules is introduced to the separation medium. In an embodiment, the method can include immobilizing or binding a target molecule to the separation medium or to a surface contacting or contained by the separation medium. In an embodiment, the target molecule can be a cell, including stem cells or cancer cells; an oligonucleotide, including DNA (deoxyribonucleic acid) and RNA (ribonucleic acid); a native cell lysate, a target overexpressing cell lysate; a native protein, a mutant protein, a peptide, an enzyme, including but not limited to cytochromes, kinases, glutaminases, phosphorylases, a ribosome, a liposome, synthetic molecules, and a nanodisc, and therein including mixtures of each, some, or all. Suitable synthetic molecules can include drugs and pollutants. In an embodiment, a nanodisc can include a lipid bilayer of phospholipids with the hydrophobic edge screened by two amphipathic proteins. Such nanodiscs are often used to study membrane proteins. In an embodiment, the target molecule is attached to a particle, including a nanotube, polymer, nanoparticle or a colloid.

In an embodiment, the target molecule can be distributed homogenously or substantially homogeneously in the separation medium along an axis of migration, wherein the axis of migration can be the direction of voltage across the separation medium. In an embodiment, the target can be distributed with an increasing or decreasing concentration gradient relative to the direction of migration to increase or decrease separation of the oligonucleotide encoded molecules. In an embodiment, the target molecule can be tethered to a particle or polymer, and then the tethered target can be mixed into a separation medium before the medium has set or gelled, and the mixture can be centrifuged to provide a concentration of gradients of tethered targets within the separation medium. One benefit of tethering targets to a polymer or particle can be selecting polymers and particles that distribute the targets in the separation medium according to designed profiles. For example, a group of homogenous target molecule can be tethered to one of two different sized particles, such that two groups or bands of the target molecule are formed when the separation medium sets or gels.

In an embodiment, the target molecule can be mixed into a separation medium before the medium has set or gelled, and the mixture can be centrifuged to provide a concentration of gradients of targets within the separation medium. Suitable centrifugation methods can include differential centrifugation, rate-zonal centrifugation, and isopycnic centrifugation. In an embodiment, concentration gradients of target materials in separation mediums can be provided by size exclusion fractionation, electrophoresis of materials, magnetic separation of magnetic targets, timed retention based on separation from other chromatographic separation techniques, or any suitable method of manipulating targets in the liquid medium.

In an embodiment, the method can include mixing an amount of a target molecule with an amount of liquid separation medium to provide a target molecule concentration in the separation medium. In an embodiment, the concentration of target molecule in the separation medium can range from about 500 μg/mL to about 5 mg/mL. In an embodiment, the method can include contacting, mixing, or binding the target molecule with a particle, polymer, or oligomer, and then adding the mixture of target molecule and particle, polymer, or oligomer to the separation medium. In an embodiment, the method can include adding the target molecule and a particle, polymer, or oligomer to the separation medium simultaneously or in any order.

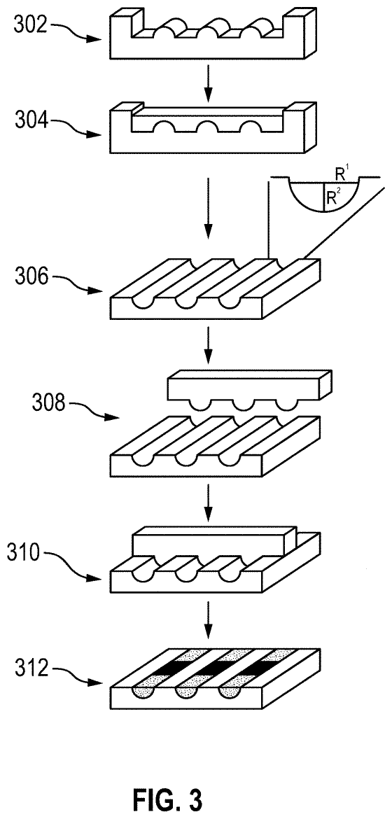

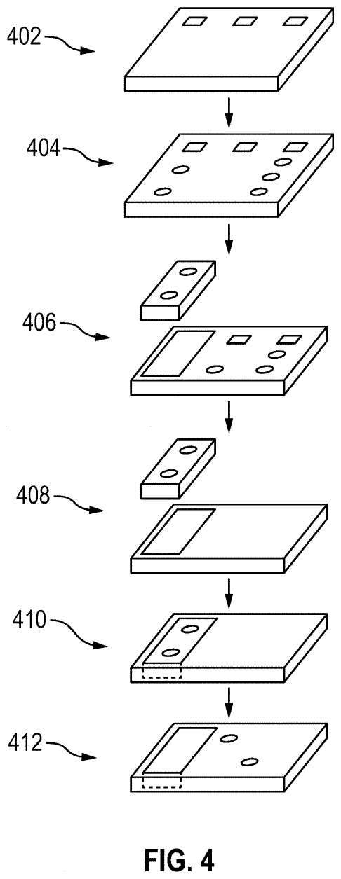

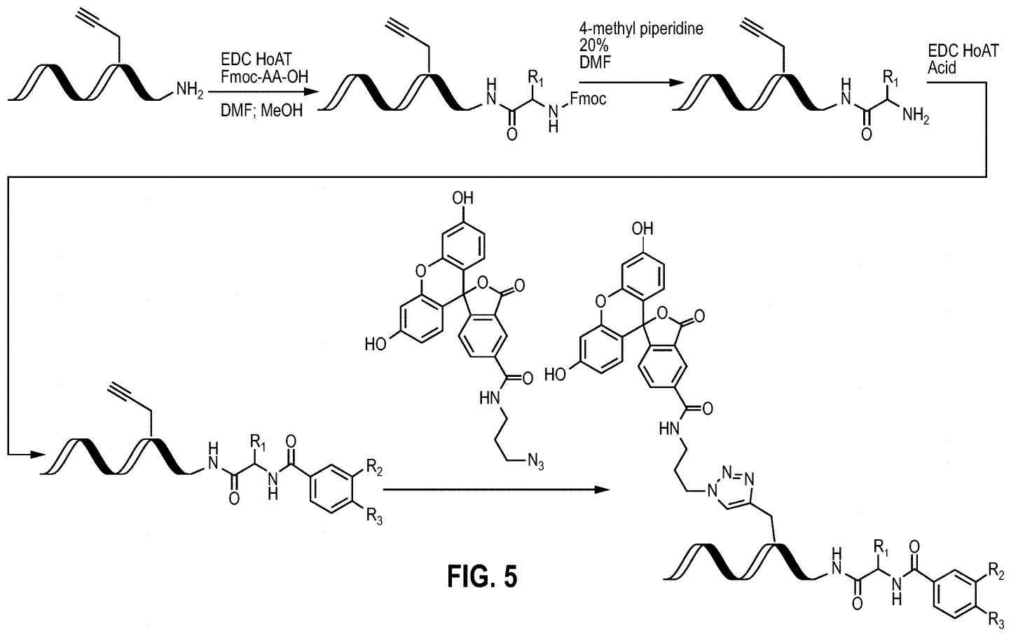

In an embodiment, the method includes providing a separation medium, wherein the separation medium contains at least one target molecule and at least one sample area or sample well. In an embodiment, the method includes adding, pouring and/or molding a separation medium onto a planar surface of an electrophoresis plate to provide a generally flat, continuous separation medium. Such a flat, continuous separation medium is illustrated in FIG. 4, 402. Referring to FIG. 3, the separation medium can be molded or shaped into lanes of separation medium as illustrated in FIG. 3. In an embodiment, the method includes providing a cast bearing lane ridges on a top surface 302; pouring a suitable polymer, such as polydimethylsiloxane (PDMS), onto the top surface of the cast bearing lane ridges and allowing it to crosslink or gel 304; orienting the molded polymer so that the lane channels face upward 306; optionally blocking sections of the mold off 308, 310; and filling the lane channels with a separation medium to form a separation medium shaped into lanes of separation 312. In an embodiment, the material blocking the section of the mold off can be removed to form sample wells 312. In an embodiment, sample wells can be cut into the separation after the separation medium has set or gelled. It has been discovered that greater resolution of oligonucleotide encoded molecules having differing activity with the target can be achieved by target-activity electrophoresis than by separations using a continuous separation medium or capillary electrophoresis. Without wishing to be bound by theory, it is believed that separations using capillary electrophoresis have the advantage that the target is not tagged, but separation is accomplished when oligonucleotide encoded molecules acquire a greater effective molecular weight when bound to target than those that are free, and thus migrate differently. However, the degree of separation achievable is limited to the differential mobility of a mobile, unbound oligonucleotide encoded molecule and a mobile, bound oligonucleotide encoded molecule. Target-activity electrophoresis can achieve greater resolution because oligonucleotide encoded molecules that bind the immobilized targets acquire an effectively infinite molecular weight, insofar as they cannot move at all while bound, whereas unbound oligonucleotide encoded molecules are still free to move at the maximum rate of the system.

In an embodiment, the method includes forming at least two different resolved oligonucleotide encoded molecules by separating the at least two different oligonucleotide encoded molecules into at least two separate locations in the separation medium, wherein the separation medium is molded or shaped into separation lanes, wherein the separation lanes have a radius of width and a radius of depth, and the radius of width R1 and radius of depth R2 can be the same or different, and can increase, decrease, or remain consistent along a length of the separation lane in the direction of migration.

In an embodiment, the method can include separating the at least two different oligonucleotide encoded molecules into at least two separate locations in the separation medium by applying a first separation treatment across the separation medium in a first direction, wherein the first separation treatment includes a first voltage protocol and a first duration. In an embodiment, the method can include harvesting the at least one resolved oligonucleotide encoded molecule by segmenting the at least one location from the separation medium in a first segmenting direction that is substantially perpendicular to the first direction to form the at least one resolved segment. In the context of direction or an axis, the term “substantially” means within 30 degrees. It is understood that the more aligned with the direction referred to, the better the results. If the first separation treatment is sufficient then no further purification or separation methods may be required. However, if the first separation treatment does not provide the desired resolution, then a subsequent second or sequential treatment can be applied. For example, after a first treatment is applied in one direction, then a second treatment may be applied by applying electrophoretic conditions in a second direction.

In an embodiment, the method includes separating the at least two different oligonucleotide encoded molecules into at least two separate locations of the separation medium by applying a second separation treatment across the separation medium in a second direction, wherein the second direction is substantially perpendicular to the first direction, wherein the second separation treatment includes a second voltage protocol and a second duration. In an embodiment, the method includes harvesting the at least one resolved oligonucleotide encoded molecule by segmenting the at least one location from the separation medium in a second segmentation direction that is substantially perpendicular to the first segmentation direction to form the at least one resolved segment. In an embodiment of the methods, the first and second separation treatments are applied while the oligonucleotide encoded molecules are maintained in the same separation medium. In an embodiment, a benefit to applying two-dimensional electrophoresis to a mixture of oligonucleotide encoded molecules can be the improved separation of the mixture of the oligonucleotide encoded molecules based on the application of different second voltage parameters, including a different voltage, a different rate of changing or ramping voltage, or pulsing voltage relative to the first voltage parameters applied. This embodiment is consistent with two-dimensional electrophoresis known in the art.

Referring to FIG. 4, in an embodiment, the method includes providing a first separation medium, wherein the first separation medium contains at least one target molecule 402; and separating the at least two different oligonucleotide encoded molecules into at least two separate locations in the separation medium by applying a first separation treatment across the separation medium in a first direction, wherein the first separation treatment includes a first voltage protocol and a first duration 404; segmenting a portion or plug of the first separation medium, including along a line, lane, or axis of separation, from the first separation medium 406; inserting or plugging the plug from the first separation medium into a sample well of a second separation medium 408, 410; and separating the at least two different oligonucleotide encoded molecules into at least two separate locations of the second separation medium by applying a second separation treatment across the separation medium in a second direction 412, wherein the second direction is substantially perpendicular to the first direction, wherein the second separation treatment includes a second voltage protocol and a second duration. In an embodiment, the method includes harvesting the at least one resolved oligonucleotide encoded molecule by segmenting the at least one location from the second separation medium in a second segmentation direction that is substantially perpendicular to the first segmentation direction to form the at least one resolved segment. In an embodiment, the first and second separation medium can be the same or different. In an embodiment, the first separation medium can contain a first target molecule and the second separation medium can contain a second target molecule, wherein the first target molecule can be the same or different from the second target molecule. In an embodiment, the concentration of the first target molecule and the second target molecule can be the same or different.

In an embodiment, the separation medium includes at least one sample area or sample well. In an embodiment, the at least one sample area is separated from an area containing the at least one target molecule, wherein the separation ranges from 1 mm to 10 cm. In an embodiment, the at least one sample area can include one or more holes, or wells, cut into the separation medium. In an embodiment, a benefit of the sample area can include a place for introducing a sample containing a mixture of at least two different oligonucleotide encoded molecules prior to electrophoresis or electrophoretic separation. In an embodiment, the method can include measuring a distance of migration from an edge of the sample area to an edge of a resolved segment, wherein the edge of the sample area is the edge in the direction of migration.

In an embodiment, a mixture of at least two different oligonucleotide encoded molecules, or a portion thereof, can be contacted to the target molecule by subjecting the sample to electrophoresis, causing the at least two different oligonucleotide encoded molecules to migrate from the sample area into contact with the at least one target molecule. The method of electrophoresis is not generally limited so long as the method is capable of contacting at least a portion of the mixture to the target molecule and/or causing the at least two different oligonucleotide encoded molecules to migrate through the separation medium. In an embodiment, the method of applying electrophoresis would not cause degradation of the oligonucleotide encoded molecules, the target molecule, or if present, a particle or polymer tethered to the target molecule.

In an embodiment, the method includes separating the at least two different oligonucleotide encoded molecules into at least two separate locations in the separation medium by applying a first separation treatment across the separation medium in a first direction, wherein the first separation treatment includes a first voltage protocol and a first duration. In an embodiment, the method includes separating the at least two different oligonucleotide encoded molecules into at least two separate locations of the separation medium by applying a second separation treatment across the separation medium in a second direction, wherein the second direction is substantially perpendicular to the first direction, wherein the second separation treatment includes a second voltage protocol and a second duration. It is understood that the separation steps can be repeated as often as desired by applying an additional separation treatment for an additional voltage protocol for an additional duration, and optionally in an additional direction. In an embodiment of the method, the first separation treatment can include a first voltage protocol of applying from about 5 V to about 150 V, including from about 30 V to about 140 V, including from about 50 V to about 120 V, for a first duration of about 1 to 50 hours, including from about 2 to about 40 hours, including about 3 to about 30 hours. In an embodiment of the method, the second separation treatment can include a second voltage protocol of applying from about 20 V to about 150 V, including from about 30 V to about 140 V, including from about 50 V to about 120 V, for a second duration of about 1 to 50 hours, including from about 2 to about 40 hours, including about 3 to about 30 hours. In an embodiment, the first treatment protocol and second treatment protocol can each independently include increasing the voltage applied by a rate from about 1 V/hr to about 5 V/hr. In an embodiment, the first treatment protocol and second treatment protocol can each independently include decreasing the voltage applied by a rate from about 1 V/hr to about 5 V/hr. It is understood that different voltage protocols may be useful for different lengths of separation medium. Generally, longer lengths require higher voltages to provide shorter separation times. In an embodiment, the method can include placing the separation medium between an anode and a cathode and applying a voltage across the separation medium or gel ranging from about 1 V/cm to about 35 V/cm. In an embodiment, the separation medium can range from about 3 cm to about 75 cm. In an embodiment, the first treatment protocol and second treatment protocol can include applying a pulsed current. In an embodiment, the first and second voltage protocol can include heating, cooling, or maintaining the separation medium to a temperature of from about 2° C. to about 60° C., including 3° C. to about 10° C., including 10° C. to about 30° C., including 30° C. to about 40° C.

In an embodiment, the method can include harvesting the at least one resolved oligonucleotide encoded molecule by segmenting the at least one location from the separation medium in a first segmenting direction that is substantially perpendicular to the first direction to form the at least one resolved segment. In an embodiment, the method can include harvesting the at least one resolved oligonucleotide encoded molecule by segmenting the at least one location from the separation medium in a second segmentation direction that is substantially perpendicular to the first segmentation direction to form the at least one resolved segment. Unless otherwise noted, the phrase “direction that is substantially perpendicular” applied to segmenting means severing or cutting at an angle measured from about 70° to about 120° from the direction of migration of the oligonucleotide encoded molecule through the separation medium. The term “segmenting” is not generally limited so long as the separation medium is divided into portions. For example, when the separation medium is a gel, then segmenting can include cutting the gel into segments. In an embodiment, provided the separation medium is a gel, the method can include freezing a gel and segmenting the frozen gel by cutting the frozen gel with a scalpel, razor or laser. Additionally, when the separation medium is a liquid, then segmenting can include withdrawing an aliquot by pipetting to form a resolved liquid segment. Referring to FIG. 2, in an embodiment of the method, a migration distance (e.g., D1, D2) for one or more segments or resolved segments of the separation medium is measured before, during or after one or more segments of the separation medium are severed or cut from the separation medium.

In an embodiment, before, during, or after the segmenting the separation medium, the at least one resolved oligonucleotide encoded molecule can be processed to allow for PCR. This step is not generally limited so long as the separation medium is changed to allow PCR. In an embodiment, provided that the separation medium is a liquid, this step can be omitted from the method. In an embodiment, removing the fraction of oligonucleotide encoded molecules from the separation segment can include soaking or wetting an isolated segment of separation medium in a solvent until at least a portion of the oligonucleotide encoded molecule diffuses from the segment into the solvent. In an embodiment, the method can include soaking a segment or resolved segment in water or a solvent to allow the OEMs to pass out of the separation medium. In an embodiment, the method can include, provided the separation medium has a melting temperature between about 20° C. to about 100° C., heating the segment or resolved segment in a buffer solution between about 20° C. to about 100° C. In an embodiment, the method can include heating the segment or resolved segment to from about 20° C. to about 100° C. and adding an enzyme capable of dissolving the gel. In an embodiment, provided the separation medium is agarose, the method can include processing the at least one resolved oligonucleotide encoded molecule to allow for PCR by adding an agarase enzyme, including alpha and/or beta agarase, including β-Agarase I (NEB in Ipswich, Mass.).

In an embodiment, a method can include amplifying the at least one encoded portion of the at least one resolved oligonucleotide encoded molecule by performing PCR on the encoding portion of the at least one resolved oligonucleotide encoded molecule to form copies of the encoding portion of the at least one resolved oligonucleotide encoded molecule. A benefit of using PCR to amplify a resolved oligonucleotide encoded molecule can include improving the signal-to-noise ratio of a resolved oligonucleotide encoded molecule of interest. A benefit of using PCR to amplify a resolved oligonucleotide encoded molecule can include learning the identity of encoded portions that were irreversibly bound to a target molecule or are otherwise difficult to remove from the separation medium due to an unforeseen reaction. The procedure for PCR can be adapted as necessary by variations known in the art.

In an embodiment, the method can include identifying or deducing the sequence, structure, or expected structure of the encoded portion of an oligonucleotide encoded molecule by sequencing the encoding portion of the oligonucleotide encoded molecule and/or, as is more likely, sequencing the encoding portion of a PCR copy of the encoding portion of the oligonucleotide encoded molecule. The procedure for sequencing the oligonucleotide encoded molecule and PCR copies of oligonucleotide encoded molecule can be adapted as necessary by variations known in the art, including applying Next-Generation DNA Sequencing, massively parallel or deep sequencing, which are all currently under research and development as these methods can be used to save time and money. In an embodiment, the method includes identifying the sequence of a fraction of copy sequences, to identify or correlate each coding region or combination of coding regions of the fraction of oligonucleotide encoded molecules to identify or correlate each positional building block of the at least one encoded portion. In an embodiment, the encoded portion of an oligonucleotide encoded molecule is identified, determined, or deduced by sequencing the encoding portion of the oligonucleotide encoded molecule or a copy sequence thereof, which can include correlating the sequence of oligonucleotides in the encoding portion with the sequence of synthetic steps that were used to synthesize the encoded portion.

In an embodiment, the method can include collecting target-activity data for the at least one resolved oligonucleotide encoded molecule by correlating the at least one location with an identity of the at least one encoded portion of the at least one resolved oligonucleotide encoded molecule. In an embodiment, the identity of the encoded portion of an oligonucleotide encoded molecule or resolved oligonucleotide encoded molecule can be correlated, matched, or associated with the migration distance measured. It is believed that oligonucleotide encoded molecules having a low activity for a target molecule will migrate quickly through the separation medium relative to oligonucleotide encoded molecules having a higher activity for a target molecule, because those with higher activities will have their progress slowed or impeded during the reaction. Further, without wishing to be bound by theory, it is believed that the oligonucleotide encoded molecule interacting with a target molecule will have a kₒₙ and k_{off}, wherein kₒₙ is the rate at which the oligonucleotide encoded molecule reacts, interacts, or associates with the target molecule and k_{off} is the rate at which the oligonucleotide encoded molecule disassociates or separates from the target molecule. In general, it is observed that OEMs having tighter affinities migrate more slowly, and OEMs having looser affinities migrate more quickly. However, it is understood that target activity will not necessarily be a factor that influences electrophoretic migration. For example, rates of electrophoresis can increase based on smaller molecular size and molecules having a higher net negative charge. These factors can be negated by introducing appropriate control molecules. Another method of isolating target activity from other electrophoretic factors can be to introduce two or more encoded portions per oligonucleotide encoded molecule. For example, an OEM having one encoded portion would be expected to have its migration slowed by one retention time. Then it stands to reason that an OEM having two or three encoded portions would have an activity 2 or 3 times greater than the OEM having one encoded portion, such that the migration of the OEM having 2-3 encoded portions would be slowed down relative to the OEM having only from 1.5 to 3 times the retention time. A benefit of the methods disclosed herein can be that the use of OEMs having multiple encoded portions can be the isolation of target activity as a factor relative to other factors for the purpose of calculating target activity data. A benefit of such a method can include the use of OEMs having multiple encoded portions to enhance the signal-to-noise ratio, such that those OEMs having only slightly different target activities can be separated or resolved on the basis of that target activity by introducing multiple encoded portions per OEM to magnify the difference in their retention times and therefore the difference in their calculated target reactivities.

In an embodiment, the method can include measuring a first distance of migration of an oligonucleotide encoded molecule from the at least one sample area and correlating the distance migrated with the identification of the encoded portion of the oligonucleotide encoded molecule. In an embodiment, the method can include measuring a second distance of migration of a second oligonucleotide encoded molecule from the at least one sample area and correlating the second distance migrated with the identification of the encoded portion of the second oligonucleotide encoded molecule. In an embodiment, the method can include calculating a relative or qualitative binding affinity of the first oligonucleotide encoded molecule for the target molecule relative to the second oligonucleotide encoded molecule by dividing the first distance by the second distance.

In an embodiment, the method can include one or more of an oligonucleotide encoded molecule having a structure according to formula (I),

G-L-B (I)

wherein
G includes the oligonucleotide comprising at least two coding regions;
B is the encoded portion containing at least two building blocks;
L is a linker that operatively links G to B; and
wherein each building block or positional building block in B is separately identified according to position by from 1 to 5 coding regions of G.

In an embodiment, the method can include one or more of an oligonucleotide encoded molecule having a structure according to formula (II),

[(B₁)_{M}-L₁]_{O}-G-[(L₂-(B₂)_{K}]_{P} (II)

wherein
G includes the oligonucleotide comprising at least two coding regions;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions of G. In an embodiment, a benefit of a method using the molecule of Formula II can include the introduction of an OEM displaying two encoded portions: (B₁)_{M} and (B₂)_{K}, which can increase the signal-to-noise ratio relative to an OEM displaying a single encoded portion, as discussed above.

In an embodiment, the method can include one or more of an oligonucleotide encoded molecule having a structure according to formula (III),

[(B₁)_{M}-L₁]_{O}-G′-[(L₂-(B₂)_{K}]_{P} (III)

wherein
G′ includes the oligonucleotide, G′ including comprising at least two coding regions and at least one hairpin;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G′;
L₂ is a linker that operatively links B₂ to G′;
O is an integer from zero to 5;
P is an integer from zero to 5;
provided that at least one of O and P is an integer from 1 to 5; and
wherein each positional building block B₁ at position M and/or B₂ at position K is identified by from 1 to 5 coding regions of G′. In an embodiment, a benefit of a method using the molecule of Formula II can include the introduction of an OEM displaying from 2 to 10 encoded portions: (B₁)_{M} and (B₂)_{K}, which can tremendously increase the signal-to-noise ratio relative to an OEM displaying a single encoded portion, as discussed above.

In certain embodiments, the present disclosure also relates to methods of forming oligonucleotide encoded molecules. In certain embodiments, the present disclosure relates to methods of separating oligonucleotide encoded molecules by using the affinity electrophoresis disclosed herein to determine the affinity of an encoded portion for a target molecule. In an embodiment, affinity electrophoresis can separate molecules based on a desired property, including but not limited to the capability of binding a target molecule, of binding to a particular region of a target molecule, of competitive or non-competitive binding to known compounds, of not binding other anti-target molecules, of not binding other closely related classes, or families, of target molecules, of being resistant to chemical changes made by an enzyme, of being resistant to chemical changes made by a family of enzymes, of being readily chemically changed by an enzyme or family of enzymes, of having degrees of water solubility, of being tissue permeable, and of being cell-permeable.

In certain embodiments, the molecule of formula (I) is an oligonucleotide encoded molecule. In an embodiment, molecules of formulas (II) and (III) are subspecies of a molecule of formula (I). In an embodiment, the molecule of formula (III) is a subspecies of a molecule of formula (II). In certain embodiments of the molecule of formula (I), G includes an oligonucleotide that is directed or selected for the synthesis of the encoded portion. In certain embodiments of the molecule of formulas (II) and (III), (B₁)_{M} and (B₂)_{K} each represent an encoded portion. In certain embodiments of the molecule of formula (I), the molecule contains an oligonucleotide portion and at least one encoded portion. It is understood that many of the structural features of the oligonucleotide in G are discussed herein in terms of their having directed or encoded the synthesis of the at least one encoded portion of the molecule of formula (I) as well as the molecular structural relationship or correlation that this synthetic process imposes on the structure of the oligonucleotide encoded molecule. It is understood that many of the structural features of the oligonucleotide in G or G′ of the molecule of formula (I) or formula (II) and/or (III), respectively, are discussed in terms of the ability of the oligonucleotide in G or G′, or a PCR copy thereof, to identify, correlate, or facilitate the deduction of the synthetic steps used to prepare the molecule of formula (I). Therefore, it is understood that there is a correlation between the sequence and/or structure of the building blocks of the encoded portion and the sequence or combination of sequences of the coding regions of the oligonucleotide portion. In an embodiment of the molecules for formulas (I) and/or (II), G includes at least one hairpin, and can be denoted as G′.

In certain embodiments of the molecule of formulas (I), (II), and (III), G or G′ includes or is an oligonucleotide. In certain embodiments, the oligonucleotide contains at least two coding regions, wherein from 1% to 100%, including from about 50% to 100%, including from about 90% to 100%, of the coding regions are single stranded. In certain embodiments, the oligonucleotide in G or G′ contains at least one terminal coding region, wherein one or two of the terminal coding regions are single stranded. In certain embodiments, the oligonucleotide in G or G′ contains at least one terminal coding region, wherein one or two of the terminal coding regions are double stranded.

The term “hairpin structure” as used in the present disclosure refers to a molecular structure that contains from 60% to 100% nucleotides by mass percent, and can hybridize to a terminal coding region of the oligonucleotide G to form G′. In certain embodiments of the hairpin structure, the hairpin structure forms a single, continuous polymer chain, and contains at least one overlapping portion (commonly called a “stem”), wherein the overlapping portion contains a sequence of nucleotides that is hybridized to a complementary sequence of the same hairpin structure. In certain embodiments of the hairpin structure, a bridge structure connects two separate oligonucleotide strands; said bridge structure may be comprised of a polyethylene glycol (PEG) polymer of between 2 and 20 PEG units, including between 3 and 15 PEG units, including between 6 and 12 PEG units. In certain embodiments of the hairpin structure, the bridge structure may be comprised of an alkane chain of up to 30 carbons, or a polyglycine chain of up to 20 units, or comprised of some other chain that bears a reactive functional group.

In certain embodiments of the molecule of formulas (I), (II), and (III), the oligonucleotide in G or G′ contains at least two coding regions, including from 2 to about 21 coding regions, including from 3 to 10 coding regions, including from 3 to 5 coding regions. In certain embodiments, if the number of coding regions falls below 2, then no combination of the coding regions would be possible. In certain embodiments, if the number of coding regions exceeds 20, then synthetic inefficiencies could interfere with accurate synthesis.

In certain embodiments of the molecule of formulas (I), (II), and (III), from about 50% to 100% of the at least two coding regions contain from about 6 to about 50 nucleotides, including from about 12 to about 40 nucleotides, including from about 8 to about 30 nucleotides. In certain embodiments, if the coding region contains less than about 6 nucleotides then the coding region cannot accurately direct synthesis of the encoded portion. In certain embodiments, if the coding region contains more than about 50 nucleotides then the coding region could become cross reactive. Such cross reactivity would interfere with the ability of the coding regions to accurately direct and identify the synthesis steps used to synthesize the encoded portion of a molecule of formulas (I), (II), and (III).

In certain embodiments of the molecule of formula (I), (II), and (III), a purpose of the oligonucleotide in G or G′ is to direct the synthesis of at least one encoded portion of the molecule of formulas (I), (II), or (III) by selectively hybridizing to a complementary anti-coding strand. In certain embodiments, the coding regions are single stranded to facilitate hybridization with a complementary strand. In certain embodiments, from 70% to 100%, including from 80% to 99%, including from 80 to 95%, of the coding regions are single stranded. It is understood that the complementary strand for a coding region, if present, could be added after steps of encoding the encoded portion of the molecule of formulas (I), (II), and (III) during synthesis.

In certain embodiments, the oligonucleotide can contain natural and unnatural nucleotides. Suitable nucleotides include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Other suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. It is understood that an oligonucleotide is a polymer of nucleotides. The terms “polymer” and “oligomer” are used herein interchangeably. In certain embodiments, the oligonucleotide does not have to contain contiguous bases. In certain embodiments, the oligonucleotide can be interspersed with linker moieties or non-nucleotide molecules.

In certain embodiments of the molecule of formulas (I), (II) (III), the oligonucleotide in G contains from about 60% to 100%, including from about 80% to 99%, including from about 80% to 95% DNA nucleotides. In certain embodiments, the oligonucleotide contains from about 60% to 100%, including from about 80% to 99%, including from about 80% to 95% RNA nucleotides.

In certain embodiments of the molecule of formulas (I), (II), and (III), the oligonucleotide in G or G′ contains at least two coding regions, wherein the at least two of the coding regions overlap so as to be coextensive, provided that the overlapping coding regions only share from about 30% to 1% of the same nucleotides, including about 20% to 1%, including from about 10% to 2%. In certain embodiments of the molecule of formulas (I), (II), and (III), the oligonucleotide in G or G′ is from about 40% to 100%, including about from 60% to 100%, including about from 80% to 100%, single stranded. In certain embodiments of the molecule of formulas (I), (II), and (III), the oligonucleotide in G or G′ contains at least two coding regions, wherein at least two of the coding regions are adjacent. In certain embodiments of the molecule of formulas (I), (II), and (III), the oligonucleotide in G or G′ contains at least two coding regions, wherein the at least two coding regions are separated by regions of nucleotides that do not direct or record synthesis of an encoded portion of the molecule of formulas (I), (II), or (III).

The term “non-coding region,” when present, refers to a region of the oligonucleotide that either cannot hybridize with a complementary strand of nucleotides to direct the synthesis of the encoded portion of the molecule of formulas (I), (II), and (III) or does not correspond to any anti-coding oligonucleotide used to sort the molecules of formulas (I), (II), and (III) during synthesis. In certain embodiments, non-coding regions are optional. In certain embodiments, the oligonucleotide contains from 1 to about 20 non-coding regions, including from 2 to about 9 non-coding regions, including from 2 to about 4 non-coding regions. In certain embodiments, the non-coding regions contain from about 4 to about 50 nucleotides, including from about 12 to about 40 nucleotides, and including from about 8 to about 30 nucleotides.

In certain embodiments of the molecule of formulas (I), (II), and (III), one purpose of the non-coding regions is to separate coding regions to avoid or reduce cross-hybridization, because cross-hybridization would interfere with accurate encoding of the encoded portion of the molecule of formulas (I), (II), and (III). In certain embodiments, one purpose of the non-coding regions is to add functionality, other than just hybridization or encoding, to the molecule formulas (I), (II), and (III). In certain embodiments, one or more of the non-coding regions can be a region of the oligonucleotide that is modified with a label, such as a fluorescent label or a radioactive label. Such labels can facilitate the visualization or quantification of molecules for formulas (I), (II), and (III). In certain embodiments, one or more of the non-coding regions are modified with a functional group or tether which facilitates processing. In certain embodiments, one or more of the non-coding regions are double stranded, which reduces cross-hybridization. In certain embodiments, it is understood that non-coding regions are optional. In certain embodiments, suitable non-coding regions do not interfere with PCR amplification of the oligonucleotide.

In certain embodiments, one or more of the coding regions can be a region of the oligonucleotide in G or G′ that is modified with a label, such as a fluorescent label or a radioactive label. Such labels can facilitate the visualization or quantification of molecules for formulas (I), (II), and (III). In certain embodiments, one or more of the coding regions are modified with a functional group or tether which facilitates processing.

In certain embodiments of the molecule of formulas (I), (II), and (III), G or G′ comprises a sequence represented by the formula (C_{N}—(Z_{N}—C_{N+1})_{A}) or (Z_{N}—(C_{N}—Z_{N+1})_{A}), wherein C is a coding region, Z is a non-coding region, N is an integer from 1 to 20, and A is an integer from 1 to 20; wherein each non-coding region contains from 0 to 50 nucleotides and is optionally double stranded. In certain embodiments of the molecule of formulas (I), (II), and (III), each or most of the coding regions contains from 6 to 50 nucleotides. In certain embodiments of the molecule of formulas (I), (II), and (III), each or most of the coding regions contain from 8 to 30 nucleotides.

In certain embodiments of the molecule of formulas (I), (II), and (III), from about 10% to 100% of the positional building blocks B₁ at position M and/or B₂ at position K correlate to a combination of from 2, 3, 4, or 5 coding regions, including from about 20% to 100%, including from about 30% to 100%, including from about 50% to 100%, including from about 70% to 100%, including from about 90% to 100%. Conversely, in certain embodiments of the molecule of formulas (I), (II), and (III), from 0 to about 90% of the positional building blocks B₁ at position M and/or B₂ at position K correlate to or are identified by a single coding region, including from 0 to about 10%, including from 0 to about 20%, including from 0 to about 30%, including from 0 to about 50%, including from 0 to about 70%.

In certain embodiments of the molecule for formulas (I), (II), and (III), B represents a positional building block. The phrase “building block” or “positional building block” as used in the present disclosure means one unit in a series of individual building block units bound together as subunits forming a larger molecule molecular structure. In certain embodiments, (B₁)_{M} and (B₂)_{K} each independently represents a series of individual building block units bound together to form a polymer chain having M and K number of units, respectively. For example, wherein M is 10, then (B)₁₀, refers to a chain of building block units: B₁₀-B₉-B₈-B₇-B₆-B₅-B₄-B₃-B₂-B₁. For example, where M is 3 and K is 2, then formula (I) can accurately be represented by the following formula:

[((B₁)₃-(B₁)₂-(B₁)₁-L₁]_{O}-G-[(L₂-(B₂)₁-(B₂)₂]_{P}.

It is understood M and K each independently serve as a positional identifier for each individual unit of B, and that the “1” or “2” of B₁ or B₂ merely serves to distinguish which chain is being referred to.

The precise definition of the term “building block” in the present disclosure depends on its context. A “building block” is a chemical structural unit capable of being chemically linked to other chemical structural units. In certain embodiments, a building block has one, two, or more reactive chemical groups that allow the building block to undergo a chemical reaction that links the building block to other chemical structural units. It is understood that part or all of the reactive chemical group of a building block may be lost when the building block undergoes a reaction to form a chemical linkage. For example, a building block in solution may have two reactive chemical groups. In this example, the building block in solution can be reacted with the reactive chemical group of a building block that is part of a chain of building blocks to increase the length of a chain or extend a branch from the chain. When a building block is referred to in the context of a solution or as a reactant, then the building block will be understood to contain at least one reactive chemical group but may contain two or more reactive chemical groups. When a building block is referred to in the context of a polymer, oligomer, or molecule larger than the building block by itself, then the building block will be understood to have the structure of the building block as a (monomeric) unit of a larger molecule, even though one or more of the chemical reactive groups will have been reacted.

The types of molecule or compound that can be used as a building block are not generally limited, so long as one building block is capable of reacting together with another building block to form a covalent bond. In certain embodiments, a building block has one chemical reactive group to serve as a terminal unit. In certain embodiments, a building block has 1, 2, 3, 4, 5, or 6 suitable reactive chemical groups. In certain embodiments, the positional building blocks of B each independently have 1, 2, 3, 4, 5, or 6 suitable reactive chemical groups. Suitable reactive chemical groups for building blocks include, a primary amine, a secondary amine, a carboxylic acid, a thioacid, a primary alcohol, a secondary alcohol, an ester, a thiol, an isocyanate, an isothiocyanate, a chloroformate, a sulfonyl chloride, a sulfonyl fluoride, a thionocarbonate, a heteroaryl halide, an aldehyde, a ketone, a haloacetate, an aryl halide, an azide, a halide, a triflate, a diene, a dienophile, a boronic acid, a boronic ester, an alpha-beta unsaturated ketone, a cyano-acrylamide, a maleimide, an alkyne, and an alkene.

Any coupling chemistry can be used to connect building blocks, provided that the coupling chemistry is compatible with the presence of an oligonucleotide. Exemplary coupling chemistry includes, formation of amides by reaction of an amine, such as a DNA-linked amine, with an Fmoc-protected amino acid or other variously substituted carboxylic acids; formation of ureas by reaction of an amine, including a DNA-linked amine, with an isocyanate and another amine (ureation); formation of a carbamate by reaction of amine, including a DNA-linked amine, with a chloroformate (carbamoylation) and an alcohol; formation of a sulfonamide by reaction of an amine, including a DNA-linked amine, with a sulfonyl chloride; formation of a thiourea by reaction of an amine, including a DNA-linked amine, with thionocarbonate and another amine (thioureation); formation of an aniline by reaction of an amine, including a DNA-linked amine, with a heteroaryl halide (SNAr); formation of a secondary amine by reaction of an amine, including a DNA-linked amine, with an aldehyde followed by reduction (reductive amination); formation of a peptoid by acylation of an amine, including a DNA-linked amine, with chloroacetate followed by chloride displacement with another amine (an SN2 reaction); formation of an alkyne containing compound by acylation of an amine, including a DNA-linked amine, with a carboxylic acid substituted with an aryl halide, followed by displacement of the halide by a substituted alkyne (a Sonogashira reaction); formation of a biaryl compound by acylation of an amine, including a DNA-linked amine, with a carboxylic acid substituted with an aryl halide, followed by displacement of the halide by a substituted boronic acid (a Suzuki reaction); formation of a substituted triazine by reaction of an amine, including a DNA-linked amine, with a cyanuric chloride followed by reaction with another amine, a phenol, or a thiol (cyanurylation, Aromatic Substitution); formation of secondary amines by acylation of an amine including a DNA-linked amine, with a carboxylic acid substituted with a suitable leaving group like a halide or triflate, followed by displacement of the leaving group with another amine (SN2/SN1 reaction); and formation of cyclic compounds by substituting an amine with a compound bearing an alkene or alkyne and reacting the product with an azide, or alkene (Diels-Alder and Huisgen reactions). In certain embodiments of the reactions, the molecule reacting with the amine group, including a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a chloroacetate, an aryl halide, an alkene, halides, a boronic acid, an alkyne, and an alkene, has a molecular weight of from about 30 to about 500 Daltons.

In certain embodiments of the coupling reaction, a first building block might be added by substituting an amine, including a DNA-linked amine, using any of the chemistries above with molecules bearing secondary reactive groups like amines, thiols, halides, boronic acids, alkynes, or alkenes. Then the secondary reactive groups can be reacted with building blocks bearing appropriate reactive groups. Exemplary secondary reactive group coupling chemistries include acylation of the amine, including a DNA-linked amine, with an Fmoc-amino acid followed by removal of the protecting group and reductive amination of the newly deprotected amine with an aldehyde and a borohydride; reductive amination of the amine, including a DNA-linked amine, with an aldehyde, or ketone, and a borohydride followed by reaction of the now-substituted amine with cyanuric chloride, followed by displacement of another chloride from triazine with a thiol, phenol, or another amine; acylation of the amine, including a DNA-linked amine, with a carboxylic acid substituted by a heteroaryl halide followed by an SNAr reaction with another amine or thiol to displace the halide and form an aniline or thioether; and acylation of the amine, including a DNA-linked amine, with a carboxylic acid substituted by a haloaromatic group followed by substitution of the halide by an alkyne in a Sonogashira reaction; or substitution of the halide by an aryl group in a boronic ester-mediated Suzuki reaction.

In certain embodiments, the coupling chemistries are based on suitable bond-forming reactions known in the art. See, for example, March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; and Coltman et al., Principles and Applications of Organotransition Metal Chemistry, University Science Books, Mill Valley, Calif. (1987), Chapters 13 to 20; each of which is incorporated herein by reference in its entirety.

In certain embodiments, a building block can include one or more functional groups in addition to the reactive group or groups employed to attach a building block. One or more of these additional functional groups can be protected to prevent undesired reactions of these functional groups. Suitable protecting groups are known in the art for a variety of functional groups (Greene and Wuts, Protective Groups in Organic Synthesis, second edition, New York: John Wiley and Sons (1991), incorporated herein by reference in its entirety). Particularly useful protecting groups include t-butyl esters and ethers, acetals, trityl ethers and amines, acetyl esters, trimethylsilyl ethers, trichloroethyl ethers and esters and carbamates.

The type of building block is not generally limited, so long as the building block is compatible with one or more reactive groups capable of forming a covalent bond with other building blocks. Suitable building blocks include but are not limited to, a peptide, a saccharide, a glycolipid, a lipid, a proteoglycan, a glycopeptide, a sulfonamide, a nucleoprotein, a urea, a carbamate, a vinylogous polypeptide, an amide, a vinylogous sulfonamide peptide, an ester, a saccharide, a carbonate, a peptidylphosphonate, an azatide, a peptoid (oligo N-substituted glycine), an ether, an ethoxyformacetal oligomer, thioether, an ethylene, an ethylene glycol, disulfide, an arylene sulfide, a nucleotide, a morpholino, an imine, a pyrrolinone, an ethyleneimine, an acetate, a styrene, an acetylene, a vinyl, a phospholipid, a siloxane, an isocyanide, a isocyanate, and a methacrylate. In certain embodiments, the (B₁)_{M} or (B₂)_{K} of formula (I) each independently represents a polymer of these building blocks having M or K units, respectively, including a polypeptide, a polysaccharide, a polyglycolipid, a polylipid, a polyproteoglycan, a polyglycopeptide, a polysulfonamide, a polynucleoprotein, a polyurea, a polycarbamate, a polyvinylogous polypeptide, a polyamide, a polyvinylogous sulfonamide peptide, a polyester, a polysaccharide, a polycarbonate, a polypeptidylphosphonate, a polyazatide, a polypeptoid (oligo N-substituted glycine), a polyether, a polythoxyformacetal oligomer, a polythioether, a polyethylene, a polyethylene glycol, a polydisulfide, a polyarylene sulfide, a polynucleotide, a polymorpholino, a polyimine, a polypyrrolinone, a polyethyleneimine, a polyacetate, a polystyrene, a polyacetylene, a polyvinyl, a polyphospholipid, a polysiloxane, a polyisocyanide, a polyisocyanate, and a polymethacrylate. In certain embodiments of the molecule for formula (I), from about 50% to about 100%, including from about 60% to about 95%, and including from about 70% to about 90% of the building blocks have a molecular weight of from about 30 to about 500 Daltons, including from about 40 to about 350 Daltons, including from about 50 to about 200 Daltons.

It is understood that building blocks having two reactive groups would form a linear oligomeric or polymeric structure, or a linear non-polymeric molecule, containing each building block as a unit. It is also understood that building blocks having three or more reactive groups could form molecules with branches at each building block having three or more reactive groups.

In certain embodiments of the molecule for formulas (I), (II), or (III), L, L₁, and L₂ each independently represent a linker. The term “linker molecule” refers to a molecule having two or more reactive groups that is capable of reacting to form a linker. The term “linker” refers to a portion of a molecule that operatively links or covalently bonds G or a hairpin structure of G′ to a building block. The term “operatively linked” means that two or more chemical structures are attached or covalently bonded together in such a way as to remain attached throughout the various manipulations the oligonucleotide encoded molecules are expected to undergo, including PCR amplification.

In certain embodiments of the molecule for formulas (II) or (III), L₁ is a linker that operatively links B₁ to G or G′, respectively. In certain embodiments of the molecule for formula (II) or (III), L₂ is a linker that operatively links B₂ to G or G′, respectively. In certain embodiments, L₁ and L₂ are each independently bifunctional molecules linking B₁ to G or G′ by, in no particular order, reacting one of the reactive functional groups of L₁ to a reactive group of B₁ and the other reactive functional group of L₁ to a reactive functional group of G or a hairpin of G′, or in no particular order, reacting one of the reactive functional groups of L₂ to a reactive group of B₂ and the other reactive functional group of L₂ to a reactive functional group of G or a hairpin of G′. In certain embodiments of the molecule for a hairpin of G′, L₁ and L₂ are each independently linkers formed from reacting the chemical reactive groups of B₁ and G or B₂ and G with commercially available linker molecules including, PEG (e.g., azido-PEG-NHS, or azido-PEG-amine, or di-azido-PEG), or an alkane acid chain moiety (e.g., 5-azidopentanoic acid, (S)-2-(azidomethyl)-1-Boc-pyrrolidine, 4-azidoaniline, or 4-azido-butan-1-oic acid N-hydroxysuccinimide ester); thiol-reactive linkers, such as those being PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]-propionamido)hexanoate)); and amidites for oligonucleotide synthesis, such as amino modifiers (e.g., 6-(trifluoroacetylamino)-hexyl-(2-cyanoethyl)-(N,N-diisopropyl)-phosphoramidite), thiol modifiers (e.g., 5-trityl-6-mercaptohexyl-1-[(2-cyanoethyl)-(N,N-diisopropyl)]-phosphoramidite, or chemically co-reactive pair modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamido)propyl-1-O-succinoyl, long chain alkylamino CPG, or 4-azido-butan-1-oic acid N-hydroxysuccinimide ester)); and compatible combinations thereof.

In certain embodiments of the molecule of formula (III), a hairpin of G′ can be designated, H₁ or H₂, wherein each hairpin independently includes from about 20 to about 90 nucleotides, including from about 32 to about 80 nucleotides, including from about 45 to about 80 nucleotides. In certain embodiments, H₁ and H₂ each independently contains 1, 2, 3, 4, 5, 6, 7, 8, 9, or 10, including from 1 to 5, including from 2 to 4, including from 2 to 3, nucleotides modified with suitable functional groups for facilitating reaction with a linker molecule, or optionally with a building block, including cases where H₁ and H₂ each independently have been synthesized using bases like, but not limited to, 5′-Dimethoxytrityl-5-ethynyl-2′-deoxyUridine, 3′-[(2-cyanoethyl)-(N,N-diisopropyl)]-phosphoramidite (also called 5-Ethynyl-dU-CE Phosphoramidite, purchased form Glen Research, Sterling Va.). In certain embodiments, H₁ and H₂ each independently include non-nucleotides that have suitable functional groups for facilitating reaction with a linker molecule, or optionally with a building block, including but not limited to 3-Dimethoxytrityloxy-2-(3-(5-hexynamido)propanamido)propyl-1-O-[(2-cyanoethyl)-(N,N-diisopropyl)]-phosphoramidite (also called Alkyne-Modifier Serinol Phosphoramidite, from Glen Research, Sterling Va.), and abasic-alkyne CEP (from IBA GmbH, Goettingen, Germany). In certain embodiments, H₁ and H₂ each independently include nucleotides with modified bases already bearing a linker, for example H₁ and H₂ each independently could be synthesized using bases like, but not limited to, 5′-Dimethoxytrityl-N6-benzoyl-N8-[6-(trifluoroacetylamino)-hex-1-yl]-8-amino-2′-deoxyAdenosine-3′-[(2-cyanoethyl)-(N,N-diisopropyl)]-phosphoramidite (also called amino-modifier C6 dA, purchased from Glen Research, Sterling Va.), 5′-Dimethoxytrityl-N2-[6-(trifluoroacetylamino)-hex-1-yl]-2′-deoxyGuanosine-3′-[(2-cyanoethyl)-(N,N-diisopropyl)]-phosphoramidite (also called amino-modifier C6 dG, purchased from Glen Research, Sterling, Va.), 5′-Dimethoxytrityl-5-[3-methyl-acrylate]-2′-deoxyUridine,3′-[(2-cyanoethyl)-(N,N-diisopropyl)]-phosphoramidite (also called Carboxy dT, purchased from Glen Research, Sterling Va.), 5′-Dimethoxytrityl-5-[N-((9-fluorenylmethoxycarbonyl)-aminohexyl)-3-acrylimido]-2′-deoxyUridine, 3′-[(2-cyanoethyl)-(N,N-diisopropyl)]-phosphoramidite (also called Fmoc-amino modifier C6 dT, Glen Research, Sterling, Va.), 5′-Dimethoxytrityl-5-(octa-1,7-diynyl)-2′-deoxyuridine, 3′-[(2-cyanoethyl)-(N,N-diisopropyl)]-phosphoramidite (also called C8 alkyne dT, Glen Research, Sterling Va.), 5′-(4,4′-Dimethoxytrityl)-5-[N-(6-(3-benzoylthiopropanoyl)-aminohexyl)-3-acrylamido]-2′deoxyuridine, 3′-[(2-cyanoethyl)-(N,N-diisopropyl)]-phosphoramidite (also called S-Bz-Thiol-Modifier C6-dT, Glen Research, Sterling Va.), and 5-carboxy dC CEP (from IBA GmbH, Goettingen, Germany), N4-TriGl-Amino 2′deoxycytidine (from IBA GmbH, Goettingen, Germany). Suitable functional groups for modified nucleotides and non-nucleotides in H₁ and H₂ include but are not limited to a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a chloroacetate, an aryl halide, a halide, a boronic acid, an alkyne, an azide, and an alkene.

In certain embodiments, one or more of the hairpin structures H₁ and H₂ can be modified with a label, such as a fluorescent label or a radioactive label. Such labels can facilitate the visualization or quantification of molecules for formula (III). In certain embodiments, one or more of the hairpin structures H₁ and H₂ are modified with a functional group or tether which facilitates processing.

In certain embodiments of the molecule of formula (III), a benefit of the hairpin structure of H₁ and H₂ is that one or both can allow for the polydisplay of multiple encoded portions at one or both ends of the molecule of formula (III). Without wishing to be bound by theory, it is believed that the polydisplay of multiple encoded portions at one or both ends of an oligonucleotide encoded molecule of the present disclosures provides improved selection characteristics under certain conditions. For example, multivalent display of encoded compounds can increase apparent affinity through avidity effects.

In certain embodiments of the molecule of formula (II) of (III), from about 10% to 100% of the positional building blocks B₁ at position M and/or B₂ at position K correlate to a combination of from 2, 3, 4, or 5 coding regions, including from about 20% to 100%, including from about 30% to 100%, including from about 50% to 100%, including from about 70% to 100%, including from about 90% to 100%. Conversely, in certain embodiments of the molecule of formulas (II) or (III), from 0 to about 90% of the positional building blocks B₁ at position M and/or B₂ at position K correlate to or are identified by a single coding region, including from 0 to about 10%, including from 0 to about 20%, including from 0 to about 30%, including from 0 to about 50%, including from 0 to about 70%.

The present disclosure relates to methods of synthesizing oligonucleotide encoded molecules, including the molecule of formulas (I), (II), and (III). In certain embodiments of a method of synthesizing a molecule of formulas (I), (II), and (III), the method uses a series of “sort and react” steps, where a mixture of oligonucleotide encoded molecules containing different combinations of coding regions are sorted into sub-pools by selective hybridization of one or more coding regions of the oligonucleotide encoded molecule with an anti-coding oligomer immobilized on a hybridization array. In certain embodiments of the method, a benefit to sorting the oligonucleotide encoded molecules into sub-pools is that this separation allows for each sub-pool to be reacted with a positional building block B, including B₁ and/or B₂, under separate reaction conditions before the sub-pools of oligonucleotide encoded molecules are combined or mixed for further chemical processing. In certain embodiments of the method, the sort and react process can be repeated to add a series of positional building blocks. In certain embodiments of the method, a benefit of adding building blocks using a sort and react method is that the identity of each positional building block of the encoded portion of the molecule can be correlated to 1, 2, 3, 4, or 5 the coding region(s) that were used to selectively separate or sort the oligonucleotide encoded molecule prior to the addition of a building block.

In certain embodiments, one or more building blocks can be added by separating an oligonucleotide encoded molecule into sub-pools using a single sorting step, reacting the oligonucleotide encoded molecule with a building block, and then remixing. In such an embodiment, the one coding region used to sort the oligonucleotide encoded molecule during synthesis would uniquely identify or correlate to the building block according to its position, because the identity of the coding region used can be correlated to the identity of the reaction used to add the building block, which would include the identity of the positional building block added.

In certain embodiments, one or more building blocks can be added by 2, 3, 4, or 5 sorting steps, reacting the oligonucleotide encoded molecule with a building block, and then remixing. In such an embodiment, the combination or series of coding regions used to sort the oligonucleotide encoded molecule during synthesis would uniquely identify or correlate to the building block according to its position, because the combination or series of coding regions used can be correlated to the identity of the reaction used to add the building block, which would include the identity or structure of the positional building block added.

In certain embodiments, the method of synthesis can be independently switched from a single sorting step (mononomial expression) or a series of sorting steps (multinomial expression), as desired. In certain embodiments of the method, the from about 10% to 100% of the positional building blocks B₁ at position M and/or B₂ at position K are added by a series of from 2, 3, 4, or 5 sorting steps, including from about 20% to 100%, including from about 30% to 100%, including from about 50% to 100%, including from about 70% to 100%, including from about 90% to 100%. If the amount of positional building blocks added is less than 10% using a series of sorting steps, then the benefits of lower costs and more efficient synthesis would not be appreciated.

It is understood that the molecules of formulas (I), (II), and (III) can include one or more coding regions that are identical between or among molecules in a pool, but it is also understood that the vast majority, if not all, of the molecules in the pool would have a different combination of coding regions. In certain embodiments of the method, a benefit of a pool of molecules having a different combination of coding regions is that the different combinations can encode for oligonucleotide encoded molecules having a multitude of different encoded portions.

In certain embodiments, the method of synthesis includes providing at least one hybridization array. The step of providing a hybridization array is not generally limited, and includes manufacturing the hybridization array using techniques known in the art or commercially purchasing the hybridization array. In certain embodiments of the method, a hybridization array includes a substrate of at least two separate areas having immobilized anti-codon oligomers on their surface. In certain embodiments, each area of the hybridization array contains a different immobilized anti-codon oligomer, wherein the anti-codon oligomer is an oligonucleotide sequence that is capable of hybridizing with one or more coding regions of a molecule of formula (I), including formulas (II) and (III). In certain embodiments of the method, the hybridization array uses two or more chambers. In certain embodiments of the method, the chambers of the hybridization array contain particles, such as beads, that have immobilized anti-codon oligomers on the surface of the particles. In certain embodiments of the method, a benefit of immobilizing a molecule of formula (I), including formulas (II) and (III) on the array, is that this step allows the molecules to be sorted or selectively separated into sub-pools of molecules on the basis of the particular oligonucleotide sequence of one or more coding regions. In certain embodiments, the separated sub-pools of molecules can then be separately released or removed from the array into reaction chambers for further hybridization steps or chemical reaction processing. In certain embodiments, the step of releasing is optional, not generally limited, and can include dehybridizing the molecules by heating, using denaturing agents, or exposing the molecules to a buffer of pH≥12. In certain embodiments, the chambers or areas of the array containing different immobilized oligonucleotides can be positioned to allow the contents of each chamber or area to flow into an array of wells for further chemical processing.

In certain embodiments, the method includes reacting the at least one building block B, including B₁ and/or B₂, with a oligonucleotide encoded molecule to form a sub-pool of molecules of formulas (II) and (III), wherein B₁ and/or B₂ is as defined above for formulas (II) and (III). In certain embodiments, the building block B₁ and/or B₂ can be added to the container before, during, or after the molecule of formulas (II) and (III). It is understood that the container can contain solvents, and co-reactants under acidic, basic, or neutral conditions, depending on the chemistry that is used to react and covalently attach the building block B₁ and/or B₂ with the oligonucleotide encoded molecule to form the molecule of formulas (II) and (III).

In certain embodiments of the method, the amplifying step includes using PCR techniques known in the art to create a copy sequence of the oligonucleotide in G or G′ of formulas (I), (II), and (III), respectively. In certain embodiments of the method, the copy sequence contains a copy of the at least two coding regions of formulas (I), (II), and (III). In certain embodiments, one benefit of amplifying the oligonucleotide in G or G′ from the at least one probe molecule includes the ability to detect which encoded portions of an oligonucleotide encoded molecule are capable of binding a target molecule, even though the oligonucleotide encoded molecule cannot easily be removed from the target molecule. In certain embodiments, a benefit of amplification is that it allows for libraries of molecules with vast diversity to be generated. This vast diversity comes at the cost of low numbers of any given molecule of formulas (I), (II), and (III). Amplifying by PCR allows identification of oligonucleotide sequences present in very small numbers by increasing those numbers until an easily detectable number is reached. Then, DNA sequencing and analysis of the copy sequence can identify or be correlated to the encoded portion of the oligonucleotide encoded molecule of formulas (I), (II), and (III) that was capable of binding the target.

The synthesis and analysis of libraries of molecules of Formula (I), (II), and (III), or recognizable variations thereof has been disclosed previously in WO 2017/218293, WO 2018/204420, and PCT/US2018/052494 (not published at the time of filing), which are incorporated by reference herein in their entirety.

### EXEMPLARY EMBODIMENTS

Embodiment 1. A method of collecting target-activity data for at least one resolved oligonucleotide encoded molecule comprising:
providing a separation medium, wherein the separation medium contains at least one target molecule;
introducing a sample containing a mixture of at least two different oligonucleotide encoded molecules to the separation medium, wherein the at least two different oligonucleotide encoded molecules include an encoding portion operatively linked to at least one encoded portion;
forming at least two different resolved oligonucleotide encoded molecules by separating the at least two different oligonucleotide encoded molecules into at least two separate locations in the separation medium;
harvesting the at least one resolved oligonucleotide encoded molecule from the at least two different resolved oligonucleotide encoded molecules by segmenting at least one location of the at least two separate locations from the separation medium to form at least one resolved segment;
processing the at least one resolved oligonucleotide encoded molecule to allow for PCR;
amplifying the at least one encoded portion of the at least one resolved oligonucleotide encoded molecule by performing PCR on the encoding portion of the at least one resolved oligonucleotide encoded molecule; and
collecting target-activity data for the at least one resolved oligonucleotide encoded molecule by correlating the at least one location with an identity of the at least one encoded portion of the at least one resolved oligonucleotide encoded molecule.

Embodiment 2. The method of any of embodiments 1 or 3-15, wherein the at least one target molecule includes at least one of a cell, an oligonucleotide, a protein, an enzyme, a ribosome, and a nanodisc.

Embodiment 3. The method of any of embodiments 1-2 or 4-15, wherein the separation medium contains at least one of a particle, a polymer, and a separation surface, and the at least one target molecule is connected to at least one of the separation medium, the particle, the polymer, and the separation surface.

Embodiment 4. The method of any of embodiments 1-3 or 5-15, wherein the particle includes a polymer particle or a metal colloid.

Embodiment 5. The method of any of embodiments 1-4 or 6-15, wherein the polymer has a molecular weight of 10% or more of a lowest weight target molecule of the at least one target molecule.

Embodiment 6. The method of any of embodiments 1-5 or 7-15, separating the at least two different oligonucleotide encoded molecules based at least one target-activity between the at least one target molecule and the encoded portion of the at least two different oligonucleotide encoded molecules.

Embodiment 7. The method of any of embodiments 1-6 or 8-15, wherein the at least one target-activity includes a chemical modification of the encoded portion of the at least one oligonucleotide encoded molecule by the at least one target molecule.

Embodiment 8. The method of any of embodiments 1-7 or 9-15, wherein
the oligonucleotide contains at least two coding regions,
the at least one encoded portion contains at least two positional building blocks,
each positional building block of the at least one encoded portion is identified by from 1 to 5 coding regions of the oligonucleotide; and
the separation medium contains a porous gel and a buffer system.

Embodiment 9. The method of any of embodiments 1-8 or 10-15, wherein the at least two different oligonucleotide encoded molecules have a structure according to formula (I),

G-L-B (I)

wherein
G includes the oligonucleotide comprising at least two coding regions;
B is the encoded portion containing at least two building blocks;
L is a linker that operatively links G to B; and
wherein each positional building block in B is separately identified according to position by from 1 to 5 coding regions of G.

Embodiment 10. The method of any of embodiments 1-9 or 11-15, wherein the at least two different oligonucleotide encoded molecules have a structure according to formula (II),

[(B₁)_{M}-L₁]_{O}-G-[(L₂-(B₂)_{K}]_{P} (II)

wherein
G includes the oligonucleotide comprising at least two coding regions;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions of G.

Embodiment 11. The method of any of embodiments 1-10 or 12-15, wherein the at least two different oligonucleotide encoded molecules have a structure according to formula (III),

[(B₁)_{M}-L₁]_{O}-G′-[(L₂-(B₂)_{K}]_{P} (III)

wherein
G′ includes the oligonucleotide, G′ including comprising at least two coding regions and at least one hairpin;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G′;
L₂ is a linker that operatively links B₂ to G′;
O is an integer from zero to 5;
P is an integer from zero to 5;
provided that at least one of O and P is an integer from 1 to 5; and
wherein each positional building block B₁ at position M and/or B₂ at position K is identified by from 1 to 5 coding regions of G′.

Embodiment 12. The method of any of embodiments 1-11 or 13-15, further comprising:
separating the at least two different oligonucleotide encoded molecules into at least two separate locations in the separation medium by applying a first separation treatment across the separation medium in a first direction,
wherein the first separation treatment includes a first voltage protocol and a first duration.

Embodiment 13. The method of any of embodiments 1-12 or 14-15, further comprising:
harvesting the at least one resolved oligonucleotide encoded molecule by segmenting the at least one location from the separation medium in a first segmenting direction that is substantially perpendicular to the first direction to form the at least one resolved segment.

Embodiment 14. The method of any of embodiments 1-13 or 15, further comprising:
separating the at least two different oligonucleotide encoded molecules into at least two separate locations of the separation medium by applying a second separation treatment across the separation medium in a second direction, wherein the second direction is substantially perpendicular to the first direction,
wherein the second separation treatment includes a second voltage protocol and a second duration.

Embodiment 15. The method of any of embodiments 1-14, further comprising:
harvesting the at least one resolved oligonucleotide encoded molecule by segmenting the at least one location from the separation medium in a second segmentation direction that is substantially perpendicular to the first segmentation direction to form the at least one resolved segment.

### Even More Exemplary Embodiments

Embodiment 1A. A method comprising:
providing a separation medium, wherein the separation medium contains at least one target molecule and at least one sample area;
introducing a sample containing a mixture of at least two different oligonucleotide encoded molecules to the sample area of the separation medium, wherein the oligonucleotide encoded molecule includes an encoding portion operatively linked to an encoded portion; and
contacting at least a portion of the mixture to the target in the separation medium by subjecting the sample to electrophoresis.

Embodiment 2A. The method of Embodiment 1A, wherein the encoding portion contains an oligonucleotide connected to at least one encoded portion,
wherein the oligonucleotide contains at least two coding regions,
wherein the at least one encoded portion contains at least two positional building blocks,
wherein each positional building block of the at least one encoded portion is identified by from 1 to 5 coding regions; and
the separation medium contains a porous gel and a buffer system.

Embodiment 3A. The method of Embodiment 1A, where in the at least one target molecule includes at least one of a cell, an oligonucleotide, a protein, an enzyme, a ribosome, and a nanodisc.

Embodiment 4A. The method of Embodiment 1A, wherein the at least two different oligonucleotide encoded molecules have a structure according to formula (I),

G-L-B (I)

wherein
G includes the oligonucleotide comprising at least two coding regions;
B is the encoded portion containing at least two building blocks;
L is a linker that operatively links G to B; and
wherein each positional building block in B is separately identified according to position by from 1 to 5 coding regions of G.

Embodiment 5A. The method of Embodiment 1A, wherein the at least two different oligonucleotide encoded molecules have a structure according to formula (II),

[(B₁)_{M}-L₁]_{O}-G-[(L₂-(B₂)_{K}]_{P} (II)

wherein
G includes the oligonucleotide comprising at least two coding regions;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions of G.

Embodiment 6A. The method of Embodiment 1A, wherein the at least two different oligonucleotide encoded molecules have a structure according to formula (III),

[(B₁)_{M}-L₁]_{O}-G′-[(L₂-(B₂)_{K}]_{P} (III)

wherein
G′ includes the oligonucleotide, G′ including comprising at least two coding regions and at least one hairpin;
B₁ is a positional building block and M represents an integer from 1 to 20; B₂ is a positional building block and K represents an integer from 1 to 20, wherein B₁ and
B₂ are the same or different, wherein M and K are the same or different;
L₁ is a linker that operatively links B₁ to G′;
L₂ is a linker that operatively links B₂ to G′;
O is an integer from zero to 5;
P is an integer from zero to 5;
provided that at least one of O and P is an integer from 1 to 5; and
wherein each positional building block B₁ at position M and/or B₂ at position K is identified by from 1 to 5 coding regions of G′.

Embodiment 7A. The method of Embodiment 1A, further comprising:
separating the at least two different oligonucleotide encoded molecules by applying a voltage across the separation medium in a first direction.

Embodiment 8A. The method of Embodiment 1A, further comprising:
isolating a fraction of oligonucleotide encoded molecules by severing the separation medium in a first severing direction that is substantially perpendicular to the first direction to form a separation segment.

Embodiment 9A. The method of Embodiment 8A, further comprising:
removing the fraction of oligonucleotide encoded molecules from the separation segment to form a recovered fraction of oligonucleotide encoded molecules;
amplifying the oligonucleotide from the recovered fraction of oligonucleotide encoded molecules to form a fraction of copy sequences,
sequencing the fraction of copy sequences to identify each coding region or combination of coding regions of the recovered fraction of oligonucleotide encoded molecules to identify each positional building block of the at least one encoded portion.

Embodiment 10A. The method of Embodiment 7A, further comprising:
separating the at least two different oligonucleotide encoded molecules by applying a voltage across the separation medium in a second direction, wherein the second direction is substantially perpendicular to the first direction.

Embodiment 11A. The method of Embodiment 10A, further comprising:
isolating a fraction of oligonucleotide encoded molecules by severing the separation medium in two directions, wherein the two directions are each independently substantially perpendicular to the first and second direction to form a rectangular separation segment.

Embodiment 12A. The method of Embodiment 11A, further comprising:
removing the fraction of oligonucleotide encoded molecules from the rectangular separation segment to form a recovered fraction of oligonucleotide encoded molecules;
amplifying the oligonucleotide from the recovered fraction of oligonucleotide encoded molecules to form a fraction of copy sequences,
sequencing the fraction of copy sequences to identify each coding region or combination of coding regions of the recovered fraction of oligonucleotide encoded molecules to identify each positional building block of the at least one encoded portion.

Embodiment 13A. The method of Embodiment 1A, wherein the separation medium contains at least one of a particle and a polymer, and the at least one target molecule is connected to at least one of the particle and the polymer.

Embodiment 14A. An electrophoretic system comprising:
a separation medium, the separation medium comprising,
a porous gel;
at least one target molecule within the porous gel; and
at least two different oligonucleotide encoded molecules within the porous gel,
   wherein the oligonucleotide encoded molecules contain an oligonucleotide connected to at least one encoded portion,
   wherein the oligonucleotide contains at least two coding regions, wherein the at least one encoded portion contains at least two positional building blocks,
   wherein each positional building block of each encoded portion is identified by from 2 to 5 coding regions.

Embodiment 15A. The electrophoretic system of Embodiment 14A, the porous gel further comprising:
at least one of a particle and a polymer, and the at least one target molecule is connected to at least one of the particle and the polymer.

### Computer Systems

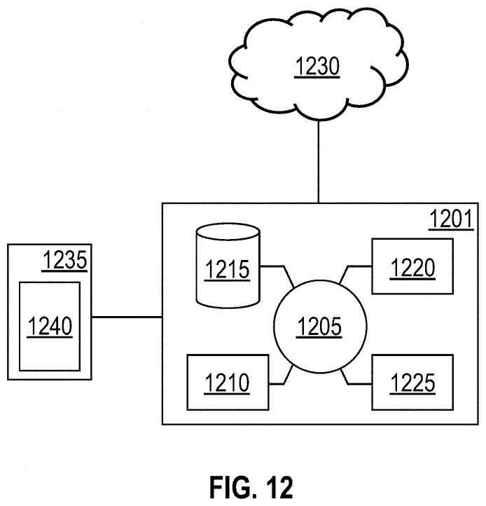

The present disclosure provides computer systems that are programmed to implement methods of the disclosure, such as, for example, collecting target-activity data for at least one resolved oligonucleotide encoded molecule. FIG. 12 shows a computer system 1201 that includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user. Examples of remote computer systems include personal computers (e.g., portable PC), slate or tablet PCs (e.g., APPLE® iPad, SAMSUNG® Galaxy Tab), telephones, Smart phones (e.g., APPLE® iPhone, Android-enabled device, BLACKBERRY®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc., shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, target-activity data for at least one resolved oligonucleotide encoded molecule. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, implement methods for collecting target-activity data for at least one resolved oligonucleotide encoded molecule.

Various embodiments of the present disclosure are illustrated by but not limited by the following examples. Those skilled in the art will recognize many equivalent techniques for accomplishing the steps or portions of the steps enumerated herein.

### Examples

### Example 1. Preparation of Synthetic Compounds Linked to DNA Oligonucleotides

It will be appreciated by those skilled in the art of DNA modification that reactions to couple activated acids to a reactive free amine allow for a large collection of compounds, or individual compounds, to be generated rapidly, and efficiently.

### Example 1A. Immobilization of DNA on SEPHAROSE® Resin

To perform chemical modification on DNA oligo linked to a reactive amine handle the DNA is first immobilized onto SEPHAROSE® resin. To each well in a 384 well filter plate (E&K Scientific, EK-2288) was added 40 uL of 1:1 DEAE SEPHAROSE®:Storage solution was added. The wells were washed on a plate vacuum manifold twice with 70 uL of water followed by two washed with 70 uL of binding buffer (10 mM AcOH in Distilled water). The plate was spun in the centrifuge for 1 minute at 2000 rpm to dry all liquid from wells. To each well was added 40 uL of binding buffer and 10 uL of 100 ng/uL of appropriate amine linked DNA oligo, the well was triturated using wide bore tips (Rainin) and allowed to incubate at RT for 10 minutes. The plate was spun into a receiver plate (greiner bio-one REF 781201) at 2000 RPM for 1 minute. The eluent in the collection plate was added to the top of the resin, the wells triturated and allowed to incubate for 5 minutes at RT. The plate was spun again into a receiver plate (2000 RPM 1 minute) and each eluent well was analyzed by nanodrop for DNA concentration to assess the capture efficiency. If measured DNA concentration was above 1 ng/uL the eluent was added to the resin a third time and incubated for an additional 5 minutes and the spin out and measurement was repeated. If the measured DNA concentration was less than 1 ng/uL the wells were washed on a vacuum manifold (3× 70 uL of binding buffer, 3× 70 uL of water, 3× 70 uL of methanol).

### Example 1B. Generation of Activated Acid General Procedure and Coupling to DNA Oligo Linked to A Reactive Amine

To couple a chemical compound containing an acid moiety to the free amine of the DNA oligomer the acid is activated in the following manner. For each acid a solution in 80:20 DMF:MeOH and concentration of 400 mM was prepared as calculated by the molecular weight of the acid. A 1 mL solution of 40 mM HoAT (5.5 mg) was prepared in 80:20 DMF:MeOH. Immediately before use a stock solution of 100 mM EDC*HCl 7.7 mg was dissolved in 400 uL of MeOH. To generate the activated acid to 15 uL of desired acid (400 mM) was added to 15 uL of HoAT (40 mM) followed by 30 uL of DMF:MeOH 80:20 and finally 60 uL of EDC*HCl solution (100 mM) was added and the mixture incubated for 5 minutes at RT.

### Example 1C. Acylation of DNA By Activated Acid General Procedure

To couple the activated acid solution to the DNA oligo linked to a reactive amine handle the immobilized DNA from Example 1A in the filter plate was washed on a vacuum manifold 3 times with 70 uL of 400 mM DIPEA in DMF:MeOH 80:20, followed by 3 washes with 70 uL of MeOH. The filter plate was then placed on a rubber stopper to prevent wells from draining. To each well was added 70 uL of the desired activated acid solution prepared in example 1B and wells triturated. The wells were sealed with metal tape seal (Corning, Cat. #6569) and allowed to incubate (RT, 1 hr). The plate was then unsealed and solution removed via vacuum manifold. The plate bottom was sealed with the rubber stopper and fresh aliquot (70 uL) of activated acid was added to the appropriate wells, triturated and the plate top sealed with metal tape seal and allowed to incubate (RT, 1 hr). After incubation the plate seals were removed, the solution removed by vacuum manifold and the wells washed (3λ, 70 uL, 80:20 DMF:MeOH).

### Example 1D. Removal of Amine Protecting Group Fluorenylmethyloxycarbonyl (Fmoc)

For DNA-linked reactive amines that were coupled with, reacted with, an activated acid (Example 1C) For wells where Fmoc protected acids were acylated was added 60 uL of 4-methyl piperidine (20% v/v, DMF) and incubated for 10 minutes. The solution was removed by vacuum manifold and wells washed (3× 70 uL DMF, 3× 70 uL MeOH, 3× 70 uL DI water). The plate was spun down to remove any traces of liquid (1 minute, 2000 RPM). This newly modified DNA oligo can either be eluted (Example 1E) or can have additional couplings performed (as per Example 1C-1D) to extend the chemical compound.

### Example 1E. Elution, Recovery and Purification of Synthetically Modified Oligonucleotides from SEPHAROSE®

After the reactions to modify the DNA linked reactive amine while immobilized on SEPHAROSE® resin the DNA compound construct may then be eluted from the wells into a collection plate for analysis and purification. To the wells was added 33 uL of elution buffer (1.5 M NaCl, 50 mM NaOH in distilled water), triturated and allowed to incubate for 5 minutes. Filter plates were placed on a receiver plate and solution collected by centrifugation (1 min., 2000 RPM). These steps were repeated two additional times to yield eluted DNA in 99 uL of elution buffer. To the collected eluent was added 2.5 uL of 1 M acetic acid (final concentration 25 mM) to neutralize 50% of the NaOH while preventing the eluent from becoming acidic. Samples were purified by Agilent 1200 HPLC on a Phenomenex Clarity 2.6 um Oligo-XT 100 A column (50×2.1 mm) using an HPLC method optimized to the particular compound-DNA construct of interest.

### Example 1F. Synthesis and Generation of Full Length Encoded Positive Control Molecules Appended to DNA

One familiar with the art will appreciate that longer DNA constructs can be generated utilizing modified primers, such as DNA oligonucleotide modified with synthesized molecules from Example 1A-1E. BCA positive controls were synthesized on a selected DNA oligonucleotide of as described. The purified Compound-DNA hybrids were used as a primer in a standard PCR using a full-length strand template specific to the identity of the compound. Briefly the template strand consisting of Za′-A(097-107)-Zbi-Bi-Zbf-Bf-Zci-Ci-Zcf-Cf-Zd-D001-Zf was added 1 uL of 10 uM Compound-Za-DNA hybrid into a 25 uL Q5 PCR reaction and as one skilled in the art will appreciate an optimized PCR program was run to generate a “full length” 234 oligonucleotide code, mimicking the length and composition of the DNA encoded library encoding portion. The product was purified using standard protocol for thermoscientific GeneJet PCR purification kit.

### Example 2 Fluorescently Label Compounds on DNA Oligonucleotides Through Orthogonal Reaction Conditions

To produce and purify compounds of known binding affinity linked to DNA, where a modified nucleotide is utilized to attach a fluorescent compound in order to visualize the DNA. It is known in the art that attachment of a fluorophore (in this example fluorescein) can enable the determination of binding affinity and can also be utilized to visualize the compound location on a surface, gel, or sample.

### 2A. Utilization of Modified DNA Code Containing a Commercially Available Alkyne Modified Oligonucleotide

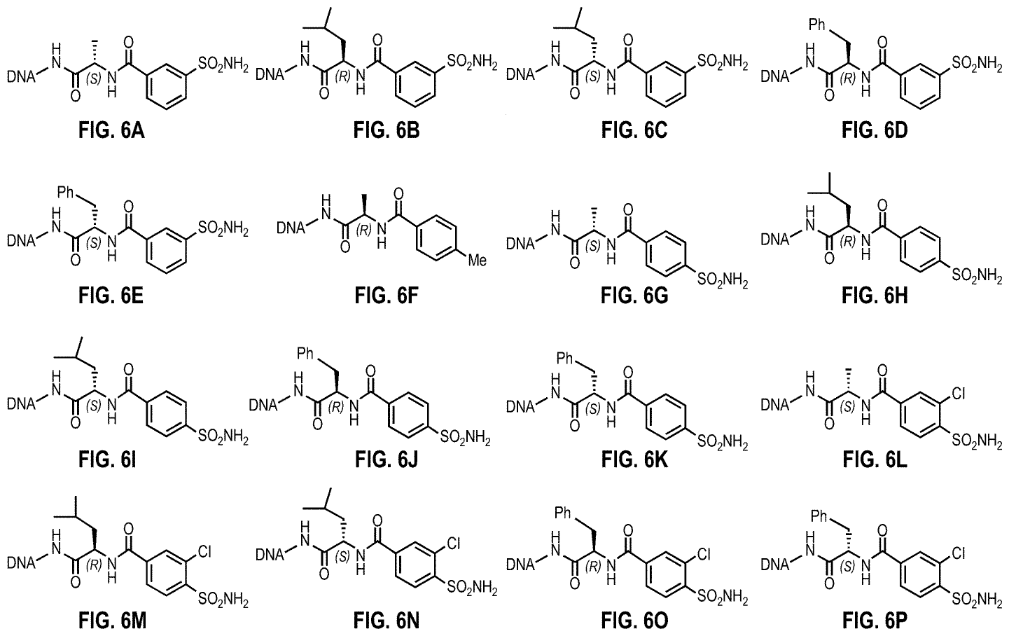

As is familiar to those with experience in the art, the utilization of an orthogonal reactive handle, in this case an alkyne, allows for synthetic modification to a distal site from the chemical compound of interest. To generate fluorescent compounds the alkyne modified DNA nucleotide (i5OctdU) oligonucleotide possessing a reactive amine linked terminus (5AmMC6) was used in the synthesis of desired compounds (shown in FIG. 6) and purified as in examples 1B-1E. After purification the sample was dried via speed-vac and dissolved in 20 uL of DI water. To this solution was added 10 uL of 7.3 mM 5-FAM azide (Lumiprobe, Cat. #C4130) followed by addition of 80 uL of freshly prepared click reaction buffer (120 mM phosphate, 1.2 mM amino guanidine, 3 mM tris(benzyltriazolylmethyl)amine, 6 mM copper sulfate, 12 mM sodium ascorbate in water) and allow to incubate (30 min., RT). After incubation 80 uL of click quench buffer (100 mM Tris, 10 mM EDTA, 0.005% tween, 0.002% SDS in Deionized water) followed by addition of 500 uL of binding buffer. To the Eppendorf tube solution was added 40 uL of DEAE resin and allowed to incubate (20 min. RT) with gentle shaking. The tube was spun down on a benchtop centrifuge for 1 minute and the supernatant removed via pipetting. The resin was resuspended in 50 uL of water and transferred to a 384 well filter plate. Wells were washed on a vacuum manifold (3× 70 uL DI water, 3× 70 uL MeOH, 1× 70 uL DMSO, 3× 70 uL methanol, 3× 70 uL water). The DNA was eluted using the procedure described above (3× 33 uL elution buffer into a collection plate). The eluent was neutralized using 10 uL of 100 mM AcOH. Samples were purified using HPLC using a standard method.

### Example 3 Binding Affinity Measurement of Fluorescent Compounds on DNA with Target in Solution Using Fluorescence Polarization

1. Positive Control Fluorescence Polarization with Soluble Protein General Procedure

FIG. 6 shows positive control compounds used.

It will be appreciated by one skilled in the art that compounds tethered to DNA covalently may possess a modified, either higher or lower, or unchanged, affinity profile as compared to the unmodified, native, compounds. To address this, the use of a common binding assay, fluorescence polarization, was utilized to assess the selected positive control molecules synthesized as tethered moieties to a DNA oligonucleotide capable of being fluorescently labeled utilizing orthogonal chemistry. The positive control compounds on DNA (as displayed in FIG. 6) were diluted individually in 1× TrisBorate buffer (45 mM, pH 8.19) to a final concentration of 20 nM in 1 mL of buffer. A 1:1 dilution of this 20 nM stock solution was made to generate a 10 nM stock solution. A 100 uM stock solution of BCA-II was made in 1× TrisBorate buffer. To a 384 well polystyrene F-bottom small volume hibase non-binding black plate (Greinerbio-one, Cat. #784900) was added 10 uL of 20 nM stock solution of the appropriate compounds in column 1. In columns 2-20 was added 10 uL of the appropriate compound 10 nM stock solution. To column 1 was added 10 uL of the 100 uM stock BCA-II protein solution, the wells were triturated and 10 uL of the well was transferred to well column 2 and mixed by pipetting this process was repeated until well 20. This procedure yields a 1:1 dilution of the stock protein concentration in each subsequent well, thereby giving a range from 50 uM to 95 pM, while the concentration of the fluorescently labeled DNA-Compound remains constant 10 nM. The plate was read in by a Spectramax M5 plate reader using excitation/emission of 485/530 and the pre-programed fluorescence polarization method. The values obtained were graphed and results fit to the hill equation to yield a binding affinity (Kd) as recorded in nanomolar values.

### Example 4 Generation of Affinity Electrophoresis Retention Lanes for Selection of Trait Positive Vs Trait Negative Molecules

### Example 4A: To Efficiently Generate the Labeling of Target Protein with Capture Moiety for Immobilization

As will be recognized by one skilled in the art, the modification of proteins is widely used and commonplace, in this example carbonic anhydrase isozyme II from bovine erythrocytes was modified with biotin to allow for the capture of target to streptavidin coated particles. To achieve this 25 mg (0.833 umoles) of carbonic anhydrase isozyme II from bovine erythrocytes (sigma C2552-25MG) was added 450 uL of PBS (pH 7.8). To this solution was added 1.5 mg (2.5 umoles, 3 equiv.) of NHS-dPeg4-Biotin (Sigma QBD10200) dissolved in 550 uL of PBS (pH 7.8). This mixture (800 uM final concentration of protein) was placed at 4° C. overnight. This biotinylated protein was used without purification in the proceeding steps.

### Example 4B: Generation of Immobilized Target onto Streptavidin Labeled Agarose Resin

The biotinylated target protein (bovine carbonic anhydrase isozyme II) was immobilized onto a resin of interest. To achieve this into a 50 mL falcon tube was added 10.6 mL of a 50% slurry of High capacity streptavidin agarose resin (Thermo #20361). The resin was washed twice with tris-borate buffer (pH 8.19) by the following procedure: The slurry was centrifuged at 1000 RPM for 1 minute and the supernatant removed by pipette, to this was added 5 mL of tris-borate buffer and the procedure repeated. After removal of the second wash 1 mL of 800 uM biotinylated bovine carbonic anhydrase, prepared as above, was added. This slurry was mixed by gentle agitation and allowed to incubate for 2 hours at room temperature. After incubation the sample was centrifuged (1000 RPM, 1 min) and the supernatant carefully removed, while being careful to allow the resin to remain “wet”.

### Example 4C: Generation of Immobilized Target onto Alternative Streptavidin Labeled Resin Particles

As can be recognized by one skilled in the art, the immobilization technique described in example 4B can readily be modified to accommodate alternative resin types. In this example streptavidin coated silica particles (Sphero SVSIP-05-5 0.4-0.6 um), streptavidin coated polystyrene particles (Sphero SVP-05-10 0.4-0.69 um) and streptavidin coated agarose particles (lower loading capacity Thermofisher 20347) were subjected to the same procedure as above to yield particles coated with the target protein.

### Example 4D Generation of the Affinity Electrophoresis Retention Agarose Mixture Utilizing Low Melt Agarose and Target Labeled Particles

To immobilize the target coated particles into a suitable porous medium for electrophoresis those familiar with the art will recognize that the temperature of sample preparation may be controlled to prevent unfolding of the target. To achieve this control and generate immobilized target particles in separation medium low melt agarose was used. To a beaker was containing 4 grams of UltraPure low melting point agarose (Invitrogen 16520) 100 mL of 0.5× tris-borate buffer (pH 8.19) was added to generate a 4% agarose solution. The beaker was microwaved on high in 1 minute intervals with brief stirring between, until all agarose was dissolved and minimal bubbles are observed. The dissolved gel was transferred to two 50 mL falcon tubes and allowed to cool and maintained at 42° C. in a heating block. Separately a 2% low melt agarose solution was prepared as described above using 2 grams of low melt agarose and 100 mL of 0.5× tris-borate buffer (pH 8.19). The sample of 5.3 mL settled high capacity streptavidin resin that had been loaded with target protein was warmed to 42° C. in the heating block. To the warmed target particulate was added 8 mL of 4% low melt agarose at 42° C. The slurry is mixed by pipette tip thoroughly, while being careful to avoid bubbles. This mixture is capable of generating 4 lanes of 9 cm affinity electrophoresis retention lane using a custom electrophoresis mold (half cylinder 4 mm height 8 mm cross section). As will be recognized the scale of this preparation can be tuned to lower or high amounts dependent on the availability of target, availability of particles, or desired lane numbers and lengths. The prepared 2% agarose solution was utilized to create the loading point for the sample within the agarose retention lane. Briefly the loading comb (dimensions described below) is placed 1 cm from the top of the gel mold and 2% agarose is added to surround the half-cylinder load points. The 2% agarose is allowed to set at room temperature and the load comb is carefully removed from the gel, this generates depressions in each lane capable of holding between 15 uL of loading solution containing the library, or desired sample.

The mold was designed in the free online software TINKERCAD®. The specifications for design were as follows; the base L×W×D 19×9×1 depth the base was bracketed with walls on two sides (19×0.5×0.8 cm). Onto the base was printed six evenly spaced (0.5 cm separation) half cylinders (see, FIG. 3, 302) with a dimension of 19×0.8×0.4 cm. The “Loading Comb”, as depicted in the image, was printed on a base with dimensions L×W×D of 8.8×1×0.4 cm. Onto the “Loading Comb base” was printed six half-cylinders 0.3 cm high and 0.6 cm wide, the spacing between half-cylinders of 0.6 cm, bracketing the half-cylinders were two squares (0.3×0.3×0.3× cm) to fit over the edge of the poured mold that forces the comb to sit centered within the prepared mold and the half cylinders centered within the half-pipes. The print was performed by UPS store utilizing a STRATASYS® UPRINT® SE Plus 3D printer with the commonly utilized ABS (acrylonitrile butadiene styrene) printing material.

To generate the resolving selections mold for lane preparation and imaging the 3d printed mold, described above, was first lightly sprayed with the aerosol SMOOTH-ON® Universal Mold Release to create an even light coating on the 3d printed mold. The terminal, open, ends of the mold were sealed using masking tape, and SMOOTH-ON® CLEARFLEX® 50 (prepared per manufacturer's instructions of weight ratio of 1:2 A:B mixed in thoroughly and sonicated for 2 minutes to remove bubbles) was poured in carefully to avoid bubbles and allowed to set overnight. After setting the clear-flex50 mold was carefully removed, generating half-cylinder lanes capable of being filled with agarose gel. The mold was washed thoroughly with water and 70% ethanol and utilized without further modification.

The resolving selections gel lane

### Example 4E. Generation of the Full Affinity Electrophoresis Retention Lane with Loading Point

In order to create the full affinity electrophoresis retention lane construct a sample loading point may be generated to allow for introduction of the sample into the porous agarose containing the retention particles loaded with target. Using a custom printed load point comb the previously prepared 2% low melt agarose is poured into mold blocked by a filter plug. The gel is allowed to cool to room temperature, to allow the agarose to set and the comb carefully removed. The load wells are inspected for any inconsistencies or holes. The comb is replaced gently into the loading well region and excess gel is cut away. Another fresh filter plug is placed at the appropriate distance from the edge of the load region (either from about 1-9 cm or any custom distance) and the target loaded particle/low melt agarose mixture pipetted into the lane and allowed to cool to RT and set to create the “capture region” of the gel. The filter plug is cut away and to the end of the “capture region” is added 2% LMP agarose to fill the lane to the end to provide additional distance for sample molecules to travel after encountering the retention “capture region”.

### Example 5. Fractionation and Separation of Known Binding Molecules Attached to DNA Based on Affinity During Electrophoresis

### Example 5A. Fluorescent Positive Control Electrophoresis Through Target Activity Electrophoresis Retention Lane

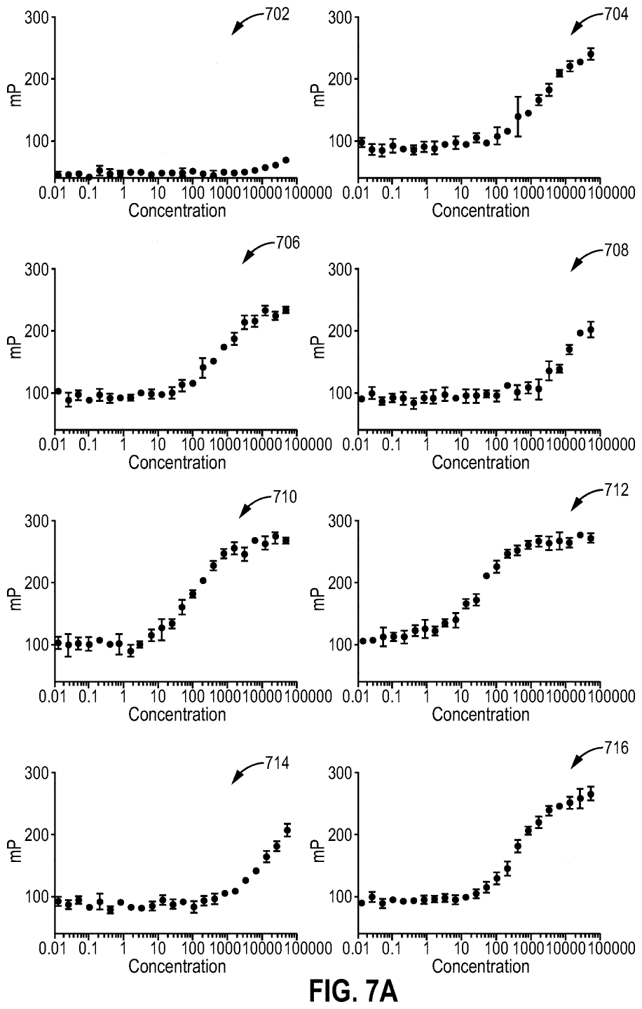

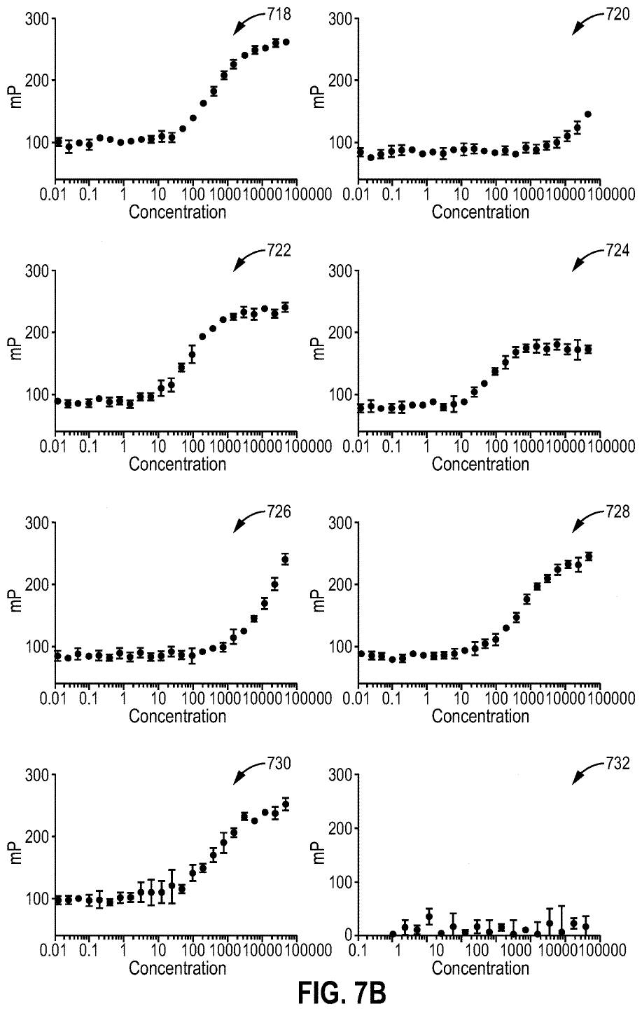

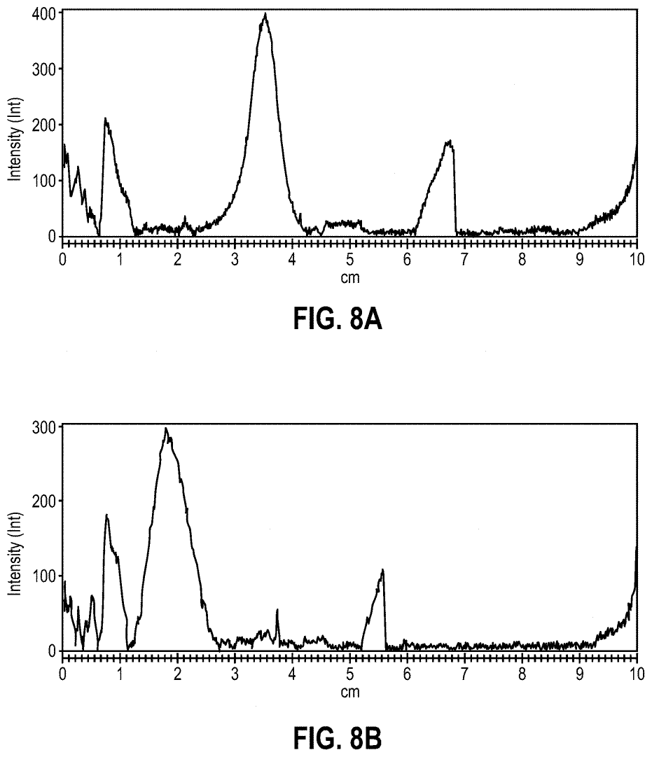

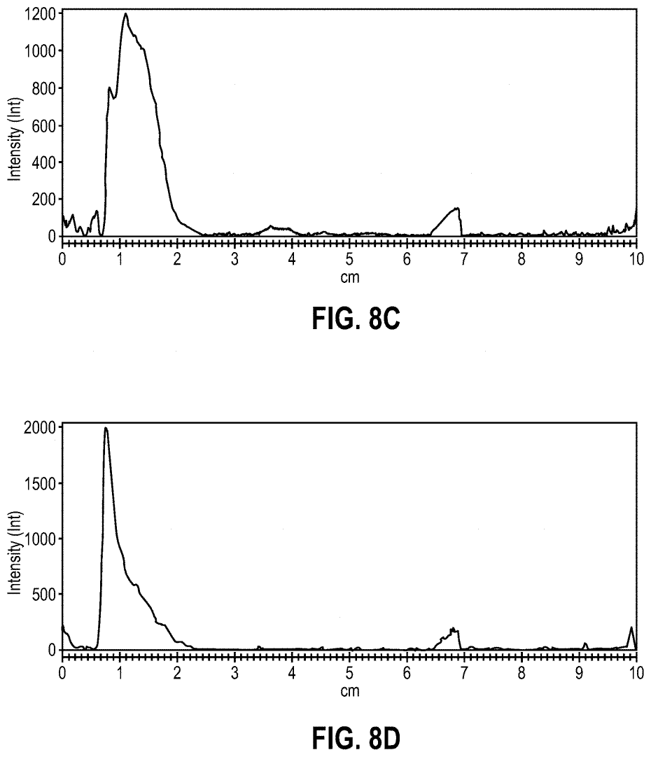

a. FIG. 7A, FIG. 7B, and FIG. 8A-D

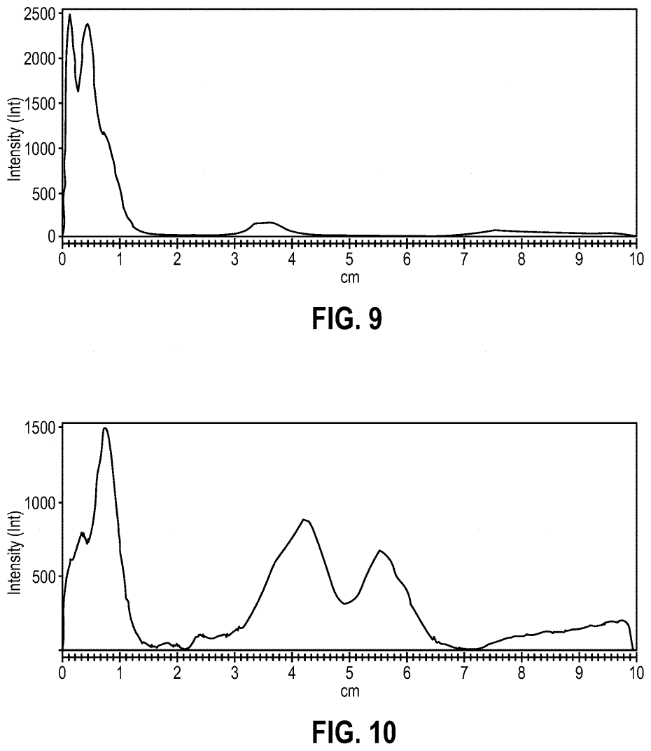

As can be appreciated by one skilled in the art of affinity chromatography the generated affinity electrophoresis retention lane will function as a fractionation dependent on the resonance time of the molecule interacting with the target to retard its motion through the gel during electrophoresis. The gel lanes with target bovine carbonic anhydrase II capture regions comprised of high loading streptavidin agarose and immobilized target were generated as described above. To these lanes was loaded 12 uL of a single positive control compound (FIG. 6) or mixtures (FIG. 8A, FIG. 8B, FIG. 8C, FIG. 8D, FIG. 9, and FIG. 10) of the fluorescently labeled positive controls (FIG. 6) with 200 ng of each compound and 2 uL of gel loading buffer. Gels were run at 40 V for 18 hours and imaged at regular intervals to track the separation progression on a Bio-Rad Gel Dock EZ Imager using a blue plate. As can be seen in FIG. 6 the individual compound experiments yielded retention factors (Rf's) that corresponded to the affinity of the synthetically modified oligonucleotide. Mixtures of these control molecules could be added and separated in the same manner (FIGS. 8A-D, 9, and 10).

### Example 5B. Fluorescent Positive Control Electrophoresis Through Affinity Electrophoresis Retention Lanes of Different Particle Types and Loading Densities

a. FIG. 10

The use of separation mediums with variable densities and particle types provides alternative retention abilities with potential for enhanced traits. To assess this, lanes were generated containing the following retention regions with captured target at 3 cm 1. Streptavidin labeled polystyrene with captured target, 2. Streptavidin labeled silica with captured target, 3. High loading streptavidin agarose, 4. Low loading streptavidin agarose (where particle density was identical to 3), 5. High loading streptavidin agarose at ¼ density to lane 3 (where total target amount was identical to 4). This explores the particle type, surface display target density and the target particle density. The experiment was performed using compounds with affinities of 362 nM (compound 10) and 7160 nM (compound 5) and fluorescein as a tracer. The electrophoresis was performed for 3 hours at 90 V.

### Example 5C. Full Length Encoding Oligonucleotide and Encoded Molecule Separation

The utility of the method lies in its ability to separate trait positive from trait negative compounds and to do so in a manner that fractionates trait positive compounds by the affinity to the target and to be sequenced to identify the encoding region and thereby the encoded compound. Before electrophoresis the gel apparatus is washed with DI water. The apparatus is filled with 2 L of 0.5× TB buffer and placed in a deli fridge in a water bath (below line of the gel apparatus) and allowed to cool to 4° C. The gel mold with lanes in loaded into the apparatus and allowed to cool for 30 minutes. To a prepared resolving selection gel (capture region of 3 cm using high loading streptavidin agarose loaded with target protein) was added 12 uL of sample containing 2 uL of purple gel loading buffer (NEB #B7024S), 100 ng of dummy library (consisting of 16 million unique library codes but not synthetically modified compounds) and 50,000 copies of each of the positive controls for Bovine Carbonic anhydrase II (Table XA), which were generated in example 1F, and encoded independently and uniquely from the dummy library sample. The sample was electrophoresed for 5 hours at 90 V.

### Example 5D Physical Partitioning of Retention Media

As can be recognized by one familiar with the art the need for clean room techniques is paramount to prevent cross contamination of samples. To achieve this after the gel is run it is transferred into a laminar flow hood. The affinity selection gel is partitioned into 44 slices of 1 mm and transferred to a sterile PCR plate. The slices are generated using a stack of 12 non-greased razor blades held between the fore fingers and depressed onto the gel slice. The PCR well location corresponds directly to the location of the gel slice.

### Example 5E Recovery of DNA Oligonucleotide from the Affinity Electrophoresis Retention Agarose Gel Lane

To generate PCR copies of the encoding DNA from each of the partitioned slices the DNA may be recovered in a form amenable to PCR amplification. To achieve this the PCR plate containing the individual gel slices generated above is heated to 95° C. for 5 minutes in a thermocycler. The Plate is spun down (2000 RPM, 1 minute) and to each well is added 3 uL of 10× B-Agarase I buffer and 5 uL of water containing 50,000 molecules of the positive control DNA sequence to yield a total volume of 30 uL. The plate is heated to 95° C. for 10 minutes again and placed in a heated block at 42° C. The sample is kept on the 42° C. heating block for 10 minutes and then to each well is added 2 uL of the B-Agarase enzyme (New England BioLabs Cat #M0392L). The plate is then allowed to incubate (lhr, 42° C.). After incubation the plate is spun down (2000 RPM, 1 minute). If necessary, the plate can be stored at −20° C. sealed. This procedure generates a sample that is no longer capable of gelling and can be amplified in the proceeding steps by PCR.

### Example 5F Indexing of Individual Gel Slices Through PCR Installation of a Specific Indexing Oligonucleotide Primer

To identify the location of the recovered DNA in reference to the affinity selection gel an indexing oligonucleotide is installed on all amplified PCR copies of the parent encoding DNA strand. To achieve this from the dissolved gel sample generated above the wells are triturated and 15 uL are transferred to a fresh 96 well plate. Each gel slice is individually indexed with zd′-D002′ through D096′-Zf indices. A total of 5 mL of master mix is prepared using Q5 High-Fidelity 2× Master Mix (NEB M0492L). To each of the wells is added 1.25 uL of the D-indexing primer (10 uM) and 1.25 uL of Illumina-Za Primer (10 uM) and run for 12 cycles with Protocol Illum_Za_T73.

### Example SG Post-Amplification with Index Installation of Illumina Za and Illumina Zf Primer Set for Sequencing

In order to sequence the encoding and indexed regions to appropriately assign the encoded compound structure a sequencing primer set may be installed. The Illumina primer set is installed using the standard PCR protocols. Briefly 2 uL is removed from each Indexed-Gel Slice well and transferred to a new 96 well PCR plate. To these wells is added 23 uL of Q5 master mix containing the Illumina primer set. The samples are run for 10 cycles using protocol Illum_Za-T73. After amplification 2 uL aliquots from each well is pooled and 1 uL of Exo-I (NEB M0293-L) per 10 uL of pooled PCR reaction is added. The combined sample is incubated at 37° C. for 30 minutes followed by heat inactivation at 80° C. for 15 minutes. The combined, exonucleased sample is purified using a GeneJet PCR clean up kit (Thermo Cat. #1(0701). The purity of the sample is assessed using a 2% agarose gel and densitometry to quantitate the amount of DNA. The purified DNA is submitted for NGS sequencing.

### Example 5H Post-Amplification with Index Installation of Illumina Za and Illumina Zf Primer Set for Sequencing

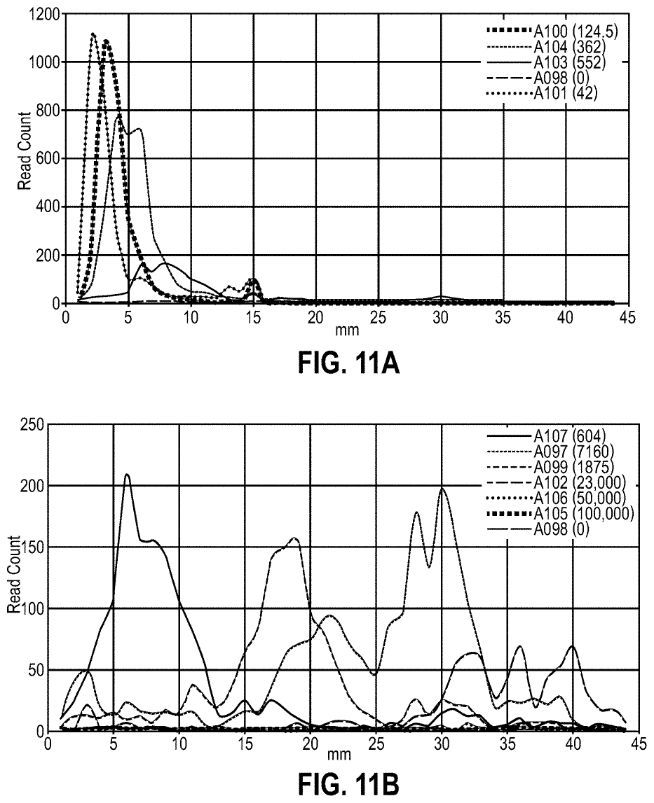

a. FIG. 11A

The DNA sample prepared in example 5F is submitted for DNA sequencing. Post processing methods isolate and identify the A097-A107 coding region, which encodes for the positive control molecules in the sample and the locational index to determine the slice from which the code originated. The sequencing counts are plotted by gel slice (FIG. 11A), where the compounds with the best affinity are highly retained as compared to compounds with a lower affinity, and can be isolated, sequenced and identified.

What is claimed is:

1. A method of determining a target-activity of at least one resolved oligonucleotide encoded molecule comprising:
providing a separation medium, wherein the separation medium contains at least one target molecule;
introducing a sample containing a mixture of at least two different oligonucleotide encoded molecules to the separation medium, wherein the at least two different oligonucleotide encoded molecules include an encoding portion operatively linked to at least one encoded portion;
forming at least two different resolved oligonucleotide encoded molecules by separating the at least two different oligonucleotide encoded molecules into at least two separate locations in the separation medium;
harvesting the at least one resolved oligonucleotide encoded molecule from the at least two different resolved oligonucleotide encoded molecules by segmenting at least one location of the at least two separate locations from the separation medium to form at least one resolved segment;
processing the at least one resolved oligonucleotide encoded molecule to allow for performing polymerase chain reaction (PCR);
amplifying the at least one encoded portion of the at least one resolved oligonucleotide encoded molecule by performing PCR on the encoding portion of the at least one resolved oligonucleotide encoded molecule; and
determining a target-activity of the at least one resolved oligonucleotide encoded molecule by processing the at least one location and an identity of the at least one encoded portion of the at least one resolved oligonucleotide encoded molecule.

2. The method of claim 1, wherein the at least one target molecule includes at least one member selected from the group consisting of a cell, an oligonucleotide, a protein, an enzyme, a ribosome, and a nanodisc.

3. The method of claim 1, wherein the separation medium contains at least one member selected from the group consisting of a particle, a polymer, and a separation surface, and the at least one target molecule is connected to at least one of the separation medium, the particle, the polymer, and the separation surface.

4. The method of claim 3, wherein the particle includes a polymer particle or a metal colloid.

5. The method of claim 3, wherein the polymer has a molecular weight of 10% or more of a lowest weight target molecule of the at least one target molecule.

6. The method of claim 1, further comprising separating the at least two different oligonucleotide encoded molecules based on at least one target-activity between the at least one target molecule and the encoded portion of the at least two different oligonucleotide encoded molecules.

7. The method of claim 6, wherein the at least one target-activity includes a chemical modification of the encoded portion of the at least one oligonucleotide encoded molecule by the at least one target molecule.

8. The method of claim 1, wherein
the oligonucleotide contains at least two coding regions,
the at least one encoded portion contains at least two positional building blocks,
each positional building block of the at least one encoded portion is identified by from 1 to 5 coding regions of the oligonucleotide; and
the separation medium contains a porous gel and a buffer system.

9. The method of claim 1, wherein the at least two different oligonucleotide encoded molecules have a structure according to formula (I),
G-L-B (I)
wherein
G includes the oligonucleotide comprising at least two coding regions;
B is the encoded portion containing at least two building blocks;
L is a linker that operatively links G to B; and
wherein each positional building block in B is separately identified according to position by from 1 to 5 coding regions of G.

10. The method of claim 1, wherein the at least two different oligonucleotide encoded molecules have a structure according to formula (II),
[(B₁)_{M}-L₁]_{O}-G-[(L₂-(B₂)_{K}]_{P} (II)
wherein
G includes the oligonucleotide comprising at least two coding regions;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G;
L₂ is a linker that operatively links B₂ to G;
O is zero or 1;
P is zero or 1;
provided that at least one of O and P is 1; and
wherein each positional building block B₁ at position M and/or B₂ at position K is identified by from 1 to 5 coding regions of G.

11. The method of claim 1, wherein the at least two different oligonucleotide encoded molecules have a structure according to formula (III),
[(B₁)_{M}-L₁]_{O}-G′-[(L₂-(B₂)_{K}]_{P} (III)
wherein
G′ includes the oligonucleotide, G′ including comprising at least two coding regions and at least one hairpin;
B₁ is a positional building block and M represents an integer from 1 to 20;
B₂ is a positional building block and K represents an integer from 1 to 20, wherein B₁ and B₂ are the same or different, wherein M and K are the same or different;
L₁ is a linker that operatively links B₁ to G′;
L₂ is a linker that operatively links B₂ to G′;
O is an integer from zero to 5;
P is an integer from zero to 5;
provided that at least one of O and P is an integer from 1 to 5; and
wherein each positional building block B₁ at position M and/or B₂ at position K is identified by from 1 to 5 coding regions of G′.

12. The method of claim 1, further comprising:
separating the at least two different oligonucleotide encoded molecules into at least two separate locations in the separation medium by applying a first separation treatment across the separation medium in a first direction,
wherein the first separation treatment includes a first voltage protocol and a first duration.

13. The method of claim 12, further comprising:
harvesting the at least one resolved oligonucleotide encoded molecule by segmenting the at least one location from the separation medium in a first segmenting direction that is substantially perpendicular to the first direction to form the at least one resolved segment.

14. The method of claim 13, further comprising:
separating the at least two different oligonucleotide encoded molecules into at least two separate locations of the separation medium by applying a second separation treatment across the separation medium in a second direction, wherein the second direction is substantially perpendicular to the first direction,
wherein the second separation treatment includes a second voltage protocol and a second duration.

15. The method of claim 14, further comprising:
harvesting the at least one resolved oligonucleotide encoded molecule by segmenting the at least one location from the separation medium in a second segmentation direction that is substantially perpendicular to the first segmentation direction to form the at least one resolved segment.
